SEQUENCE LISTING (4 sequences)

SEQ ID NO: 1 - protein, 396 aa, Rattus norvegicus
MELDHMTTGGLHAYPAPRGGPAAKPNVILQIGKCRAEMLEHVRRTHRHLLTEVSKQVERELKGLHRSVGKLENNLDGYVPTGDSQRWKKSIKACLCRCQETIANLERWVKREMHVWREVFYRLERWADRLESMGGKYPVGSEPARHTVSVGVGGPEPYCQEADGYDYTVSPYAITPPPAAGELPEQESVGAQQYQSWVPGEDGQPSPGLDTQIFEDPREFLSHLEEYLRQVGGSEEYWLSQIQNHMNGPAKKWWEFKQGSVKNWVEFKKEFLQYSEGTLSREAIQRELDLPQKQGEPLDQFLWRKRDLYQTLYVDAEEEEIIQYVVGTLQPKFKRFLRHPLPKTLEQLIQRGMEVQDGLEQAAEPSVTPLPTEDETEALTPALTSESVASDRTQPE

SEQ ID NO: 2 - protein, 396 aa, Homo sapiens
MELDHRTSGGLHAYPGPRGGQVAKPNVILQIGKCRAEMLEHVRRTHRHLLAEVSKQVERELKGLHRSVGKLESNLDGYVPTSDSQRWKKSIKACLCRCQETIANLERWVKREMHVWREVFYRLERWADRLESTGGKYPVGSESARHTVSVGVGGPESYCHEADGYDYTVSPYAITPPPAAGELPGQEPAEAQQYQPWVPGEDGQPSPGVDTQIFEDPREFLSHLEEYLRQVGGSEEYWLSQIQNHMNGPAKKWWEFKQGSVKNWVEFKKEFLQYSEGTLSREAIQRELDLPQKQGEPLDQFLWRKRDLYQTLYVDADEEEIIQYVVGTLQPKLKRFLRHPLPKTLEQLIQRGMEVQDDLEQAAEPAGPHLPVEDEAETLTPAPNSESVASDRTQPE

SEQ ID NO: 3 - DNA, 3032 bp, Rattus norvegicus
agtgctctggcgagtagtcctccctcagccgcagtctctgggcctcttcagcttgagcggcggcgagcctgccacactcgctaagctcctccggcaccgcgcacttgccactgccactgccgcttcgcgcccgctgcagccgccggctctgaatccttctggcttccgcctcagaggagttcttagcctgtcccgaaccgtaaccccggcgagcagatggagctggaccatatgacgaccggcggcctccacgcctaccctgccccgcggggtgggccggccgccaaacccaatgtgatcctgcagattggtaagtgccgagctgagatgctggagcacgtacggaggacccaccggcatctgttgaccgaagtgtccaagcaggtggagcgagagctgaaagggttgcacaggtcggtgggcaagctggagaacaacttggacggctatgtgcccacgggcgactcacagcgctggaagaagtccatcaaggcctgtctctgccgctgccaggagaccatcgccaacctggagcgctgggtcaagcgtgagatgcacgtgtggagggaggtcttctaccgtctggagaggtgggccgaccgcctggagtccatgggcggcaagtacccagtgggcagcgagccggcccgccacactgtctctgtaggtgtggggggtccagagccctactgccaggaagctgatggctacgactacactgttagcccctatgccatcaccccgccacctgccgcaggagagctgcctgagcaggagtcagttggggctcagcaataccagtcttgggtgccaggtgaggatgggcaaccaagcccaggtctggatacccagatctttgaggacccacgggagttcctgagccacctggaagagtacctgcggcaggtgggtggctctgaagaatattggctgtcccagatccagaaccacatgaatgggccagccaagaagtggtgggagttcaaacagggctcggtgaagaactgggtggagttcaagaaggagtttctgcagtacagtgagggtacgctctcccgcgaagccattcagcgggagctggacctgccacagaagcagggtgagccacttgaccagttcctctggcgtaagcgggacctgtaccagacactgtatgtggacgctgaggaggaggagatcattcagtatgtggtgggcaccctgcagcccaagttcaagcgctttctgcgccacccacttcccaagaccctggagcagctcatccagaggggcatggaagttcaggacggcctggagcaggcagctgagccttctgtcacccctctgcccacagaggatgagactgaggcactcacgcctgctcttaccagcgagtcagtagccagtgacaggacccagcctgaatagaggggccagcccagggtccccagcctgcctgccacacccagtctgtggcttttgtcaactaggacttgattgagctggggctgacacccaaggggatgccctgtccagccagacaccttctcacccactggcctgactcacaactgccacacaaccatgattcatggacatcaagaagcccctctcccatagggctcccacctgccacctacccctcacctgtctgccctagtcctggccctgtctccagtggcctcaccctctacactctcagaccatcacagaacacctttggcttcctcattctgcatcagtgtccagggccctttgggtagtcaagaaatcaagtgtctgaaaggcaatgaaaagtaggcaccaaacccaaggggcatcccagggcagatgctaaagcagaatcagagatggccgaaggaacctctacttccggggatgcagcccgctcctacagacacagcagatccagctggtgccctacctgcctcccagagcaactggccagtcttgggcagcatagctcccctctcagggtgagctgaagcagcagacctgacgcgctggcgcctcctggcccccagcagtgattcataccagtgaagaaaagcagacttcggctccatgactcagccatgccaggcggagggtcccagaggggctgagtcctcagccccagctgaggcagcagctggagtcttcagagccaggtgaatgacaccaggtctcaagctgctgagaagtctttccggccatgtctggaaggggtaccaccccagcaccagcaccgtcccctcctctcttgaagctgcctgcacagaggttccaagacactttcaaggcagagaaaataggattacaaagaggaggtgccttggcagagggcagcacccagctcagcctcagagctgaaggtgaagacaagccagcgtgaaaccccgggtctgccacgaatgcccgctccgctggccactcaccagctgcctgccacaagccactgcagcttgagcagggtctgtgccctctcagcacagagcccagttcgctgcgtggcctttggcccccgccagaaccttgcaggagccttaaggttcgggccctagcccagcctgaccttacctgctgtgccctgcctgctggtcaagtccagtcccaggagaccccatgccttggctcctaggctgttccaggcacttccctgacctgccgggtgattgcccagctggaacctcatccacaccccagcaccaaccacctcgtgttggtaactgctcgtgtctgtagtctgagtaggccatgttgaggttcctccatctgcctggtccattggtgttctgagaccagttccactgctgttctgacagatcccccaccctgtgcccctgccagcccccacaggtttatttttgcacataaaccatgacccatactaatttggctagctctggggactagggagaccctggagatctcaagagtgtggctatcccctattttcaccaagccttcaatatccagccaggccatctggcccacaccatcttacctcaaagacagacatatatatatatatacatatatatgattttgttaataaaactatgaaatttaaa

SEQ ID NO: 4 - DNA, 2931 bp, Homo sapiens
cgcgtgggccgcagcagccgagccggacctgcctccccgggcgtgctccgccggccccgccgccggcccgcagcgacagacaggcgctccccgcagctccgcacgggacccaggccgccggaccccagcgccggaccaccgtccgtccgccccgaggagtttgccgcctgccggagcacctgcgcacagatggagctggaccaccggaccagcggcgggctccacgcctaccccgggccgcggggcgggcaggtggccaagcccaacgtgatcctgcagatcgggaagtgccgggccgagatgctggagcacgtgcggcggacgcaccggcacctgctggccgaggtgtccaagcaggtggagcgcgagctgaaggggctgcaccggtcggtcgggaagctggagagcaacctggacggctacgtgcccacgagcgactcgcagcgctggaagaagtccatcaaggcctgcctgtgccgctgccaggagaccatcgccaacctggagcgctgggtcaagcgcgagatgcacgtgtggcgcgaggtgttctaccgcctggagcgctgggccgaccgcctggagtccacgggcggcaagtacccggtgggcagcgagtcagcccgccacaccgtttccgtgggcgtggggggtcccgagagctactgccacgaggcagatggctacgactacaccgtcagcccctacgccatcaccccgcccccagccgctggcgagctgcccgggcaggagcccgccgaggcccagcagtaccagccgtgggtccccggcgaggacgggcagcccagccccggcgtggacacgcagatcttcgaggaccctcgagagttcctgagccacctagaggagtacttgcggcaggtgggcggctctgaggagtactggctgtcccagatccagaatcacatgaacgggccggccaagaagtggtgggagttcaagcagggctccgtgaagaactgggtggagttcaagaaggagttcctgcagtacagcgagggcacgctgtcccgagaggccatccagcgcgagctggacctgccgcagaagcagggcgagccgctggaccagttcctgtggcgcaagcgggacctgtaccagacgctctacgtggacgcggacgaggaggagatcatccagtacgtggtgggcaccctgcagcccaagctcaagcgtttcctgcgccaccccctgcccaagaccctggagcagctcatccagaggggcatggaggtgcaggatgacctggagcaggcggccgagccggccggcccccacctcccggtggaggatgaggcggagaccctcacgcccgcccccaacagcgagtccgtggccagtgaccggacccagcccgagtagagggcatcccggagcccccagcctgcccactacatccagcctgtggctttgcccaccaggacttttgagctggggctgactcctgcaggggaagccctggtccagctgggtgccccctcgagctccgggcggactcgcacacactcgtgtcatccagatgtgagcaccgcacccagcggcaaagagccctcccccctgcagggctccacccatcaccctccctccgtctgtctttccggcctggaccccaccctccacactctcaggccatcacagaacaccccagcttcctcattctgctacaacacccaggccctctggacatccagaaaaccaagtgtccggatggcaggggccagcggccaccaagctcatgggacacccagagcagaagctagggcagagccaatgctgagggagcctcgacttccggcgccgccgccctctcccggcatccgcagagccagctgacgccctccctgcctcccagggcagctggccagcctcgggcagcgcggccccctcctcccaggggagagtagaagtcgcacacgcagcagagcagacctgatgtcccggtgcttcctggcccctcagctccagtgattcacgcccgcctggagaagaatcagagctcagctcatgactcacccatggcaggcggagggtcccagaggggctgagtcctcaaatccggctgaggcagcagctggcaccatcagagccaggagagtgacaacaggtctcaaggttcccacaaagtctttgctgctgtgctgggcaccacccacccctcaccttgcaggctgcctgcgtgggaggcgaagtcccaggacagcccagaggggggctacagagaggagtcggctgcagcagagggcaggagccccagcttagccctgagcgccagcgcgaggaccagggcctgccactaagcccgccccgctggccgccagctgcccgtccccagagccactgcagcaggagtcgggccctgcctccctcccagcagggaaaccccgcccgctgccaggccatcctctctgccagaggctttcatgagccccaaggctggggccacagctcctacccctgcccagcagccctgagctcagctgcaggaaggacatcccagaagccatggctcctggggcgcttccaggcattctgccctgccccgacaccagaaccctggtgctggtgggccactagcgtctgcagcctaagcaggtgctggctcagggttcatcattctgccttgtccactgggggaccagccctgcagaccactctgacaagtcttcagcccacaccttgccagccccacagattttatttttgcacataagccataaccaatcctcaaggctggcacaggctttggggaagccctggagcctgtgaagaccctggaaacctcatgaggctgtggccaacccctgccccttgccccacacagaccaggccttaaatgtcggtccaggccctgtgcaccttaccccagagacagactctttttgtaagattttgttaataaaacactgaaacttc

(19) United States
(12) Patent Application Publication
(10) Pub. No.: US 2021/0189432 A1
(43) Pub. Date: Jun. 24, 2021

(54) COMPOSITIONS AND METHODS OF USE OF ARC CAPSIDS

(71) Applicant: University of Utah Research Foundation, Salt Lake City, UT (US)

(72) Inventors: Jason D. Shepherd, Salt Lake City, UT (US); Cameron Day, Salt Lake City, UT (US); Elissa Pastuzyn, Salt Lake City, UT (US)

(21) Appl. No.: 16/610,408

(22) PCT Filed: May 10, 2018
(86) PCT No.: PCT/US2018/032105

§ 371 Date: Nov. 1, 2019

Related U.S. Application Data
(60) Provisional application No. 62/504,370, filed on May 10, 2017.
(60) Provisional application No. 62/543,130, filed on Aug. 9, 2017.

(51) Int. Cl. C12N 15/88; C12N 7/00; C12N 15/113; C07K 14/005

(52) U.S. Cl. CPC C12N 15/88; C12N 7/00; C12N 15/113; A61K 48/00; C12N 2310/14; C12N 2310/122; C07K 14/005

(57) ABSTRACT

Disclosed are recombinant Arc capsids. Disclosed are vectors comprising a nucleic acid sequence capable of encoding an Arc protein. Disclosed are cells comprising vectors comprising a nucleic acid sequence capable of encoding an Arc protein. Disclosed are methods of delivering mRNA to a cell comprising administering an Arc capsid to a cell, wherein the Arc capsid comprises an mRNA of interest. Disclosed are methods of delivering mRNA to a cell comprising administering any one of the disclosed vectors to a cell; and administering a mRNA of interest to the cell; wherein the nucleic acid sequence encodes an Arc protein within the cell and Arc capsids are formed, wherein the Arc capsids encapsulate the mRNA of interest. Disclosed are methods of forming Arc capsids comprising administering a vector comprising a nucleic acid sequence capable of encoding an Arc protein to a solution comprising cells, wherein the nucleic acid sequence encodes an Arc protein within the cells and Arc capsids are formed.

### REFERENCE TO SEQUENCE LISTING

The Sequence Listing submitted Feb. 26, 2020 as a text file named “21101_0352 U2_Updated_Sequence_Listing.txt,” created on Feb. 25, 2020, and having a size of 27,492 bytes is hereby incorporated by reference pursuant to 37 C.F.R. § 1.52(e)(5).

### BACKGROUND

The neuronal gene Arc is essential for long-lasting information storage in the mammalian brain, mediates various forms of synaptic plasticity, and has been implicated in neurodevelopmental disorders. However, little is known about Arc's molecular function and evolutionary origins. New studies suggest that Arc is derived from a retrotransposon with homology to the Gag polyprotein that is common to retroviruses. Arc biochemistry exhibits similar molecular properties of retroviruses.

### BRIEF SUMMARY

Disclosed are vectors comprising a nucleic acid sequence capable of encoding an Arc protein.

Disclosed are recombinant Arc capsids. Disclosed are Arc capsids conjugated to a labeling moiety or a targeting moiety or both.

Disclosed are cells comprising vectors comprising a nucleic acid sequence capable of encoding an Arc protein.

Disclosed are compositions comprising an Arc capsid and a pharmaceutically acceptable carrier.

Disclosed are methods of delivering mRNA to a cell comprising administering an Arc capsid to a cell, wherein the Arc capsid comprises an mRNA of interest.

Disclosed are methods of delivering mRNA to a cell comprising administering any one of the disclosed vectors to a cell; and administering a mRNA of interest to the cell; wherein the nucleic acid sequence encodes an Arc protein within the cell and Arc capsids are formed, wherein the Arc capsids encapsulate the mRNA of interest.

Disclosed are methods of delivering mRNA to a subject comprising administering one or more of any one of the disclosed cells to a subject in need thereof.

Disclosed are methods of forming Arc capsids comprising administering a vector comprising a nucleic acid sequence capable of encoding an Arc protein to a solution comprising cells, wherein the nucleic acid sequence encodes an Arc protein within the cells and Arc capsids are formed.

### BRIEF DESCRIPTION OF THE DRAWINGS

The accompanying drawings, which are incorporated in and constitute a part of this specification, illustrate several embodiments of the disclosed method and compositions and together with the description, serve to explain the principles of the disclosed method and compositions.

FIGS. 1A-1D. Arc forms virus-like capsids via a conserved retroviral Gag CA domain. (A) Maximum likelihood phylogeny based on an amino acid alignment of tetrapod Arc, fly dArc1, and Gag sequences from related Ty3/gypsy retrotransposons. Schematics of Gag-only Arc genes and Ty3/gypsy elements are included to the right of the tree. In lineages without Arc genes, the most closely related sequences to Arc are Gag-pol poly-proteins flanked by long terminal repeats (LTRs) as expected in bona fide Ty3/gypsy retrotransposons. (B) (top) Representative negative stain EM images of full-length purified rat Arc (prArc) protein (1 mg/mL, 42,000×). (i-iv) Magnified view of boxed particles. Scale bars=30 nm. Representative cryo-EM images of prArc (2 mg/mL, 62,000×). (v-vii) Magnified images of Arc capsids showing the double-layered capsid shell. Scale bars=30 nm. (bottom) Dynamic light scattering analysis of prArc capsids. The weighted size distribution profile is represented as a histogram of the number of particles. (C) Schematic of Arc protein with the predicted matrix (MA) (orange), CA-NTD (green), and CA-CTD (blue) domains. Also depicted: ΔCTD deletion mutant and the CA domain constructs. Representative negative stain EM images of purified GST, prArc, the Drosophila Arc homologue dArc1, prArc-ΔCTD, and CA-prArc (all 1 mg/mL, 20,000×). Inset scale bars=50 nm. (bottom) Quantification of capsid formation. Fully formed capsids include spherical particles that are between 20-60 nm and have clear double shells, while partially formed capsids do not have clear double shells (scale bars=100 nm). Data is the average of 3 independent experiments±SEM using 3 different prArc preparations. ***p<0.001, two-way ANOVA with post hoc t-tests. (D) (top) To determine properties of Arc capsid stability, prArc was exchanged into buffers with increasing molar concentrations of salt and examined by negative stain EM. Arc capsids were counted manually and quantified in each buffer condition at a protein concentration of 1.5 mg/mL. Data is the average of 3 independent experiments ±SEM using different prArc preparations. **p<0.01, Student's t-test. (bottom) Representative EM images of prArc under OM NaCl and 0.5M NaPO4 conditions.

FIGS. 2A-2E shows that Arc protein interacts with mRNA. (A) (left) qRT-PCR of Arc mRNA and the bacterial mRNA asnA from prArc (right) qRT-PCR of Arc and asnA mRNA from total bacteria lysate. Data presented as the mean±SEM normalized to the average of the asnA group (Student's t-test, n=3 independent protein preparations, *p<0.05). (B) Protein preparations were treated with or without RNase A for 15 min and qRT-PCR was performed. RNase treatment did not affect Arc and asnA mRNA levels (paired t-test, n=5 independent protein samples), but significantly degraded exogenous/free GFP mRNA (paired t-test, n=3 independent samples, *p<0.05). Data presented as ±SEM normalized to the average of the untreated group. (C) (top) Representative Western blots of Arc protein that was immunoprecipitated (IP) from WT mouse cortical tissue using an Arc or IgG antibody. Input (I)=10% total lysate. (bottom, left) Quantification of Arc protein IP, showing significant enrichment of Arc protein using an Arc antibody. (bottom, right) qRT-PCR was performed on the eluted fractions from the IP. Arc mRNA was specifically pulled down in the IP (two-way ANOVA with repeated measures and Sidak's multiple comparisons: Arc+Arc vs. Arc+IgG, p=0.01; Arc+Arc vs. GAPDH+Arc, p=0.013; Arc+Arc vs. GAPDH+IgG, p=0.011). Data presented as the mean±SEM normalized to the average of the IgG group. (D) qRT-PCR of Arc mRNA from prArc and prArc (RNA−). There was significantly less Arc mRNA in the prArc(RNA−) preparations. Presented as the mean±SEM normalized to the average of the prArc group (Student's t-test, n=3 independent samples, *p=0.05). (E) (left) Representative negative stain EM images of prArc, prArc(RNA−), and prArc(RNA−) incubated with 7.3% (w/w) GFP mRNA at RT for 2 h (0.25 mg/mL, 15,000×). Fully formed capsids are indicated by red arrows (scale bars=100 nm). (right) Capsids were quantified as in FIG. 1C. Data is presented as the average of 6 images from each condition ±SEM. ***p<0.001, unpaired t-test.

FIGS. 3A-3F shows that Arc is released from cells in extracellular vesicles. (A) HEK cells in 10-cm dishes were transfected with full-length rat WT myc-Arc and media collected 24 h later. Representative Western blots (n=3 independent experiments) show Arc protein in total cell lysates (cells) and the EV fraction purified from cell media in Arc transfected (+) and untransfected (−) cells. ALIX was used as an EV fraction marker. Ponceau stain was used to visualize the total amount of protein in each lane. (B) HEK293 cells were transfected with myc-Arc-WT or myc-Arc-ΔCTD and media collected 24 h later. Representative Western blots (n=3 independent experiments) show Arc protein in total cell lysates (cells) and the EV fraction from cell media. Arc levels in the EV fraction were normalized to Arc protein levels in the cell lysate for each experiment and data is presented normalized to WT levels (n=3). *p<0.05, Student's t-test. (C) HEK EV fractions were untreated (control) or treated with RNase (n=6 independent cultures) prior to RNA extraction. qRT-PCR was used to measure Arc mRNA levels and data is presented as the mean±SEM normalized to the average of the untreated group. Paired t-test. (D) Media was harvested from DIV15 cultured cortical neurons obtained from WT and Arc KO mice after 24 h incubation and the EV fraction was purified from collected media. Blots indicate levels of Arc, ALIX, and actin from supernatant (S)/soluble fraction and pellet (P)/insoluble fraction for total cellular lysate (cells). (S)/last wash of the ultracentrifugation purification protocol and final pellet (P)/EV fraction for purified EV fraction (EVs). 2.5% of S and P were loaded for cellular lysates. 5% of S and P were loaded for the EV fraction. (E) RT-PCR using Arc and GAPDH primers was performed on WT or KO mouse cortical tissue, mouse cortical DIV15 WT or KO neurons (cells), and EVs purified from media collected from WT or KO cultured neurons. Arc mRNA was present in all three preparations, while GAPDH mRNA was absent from EVs. (F) (top) Immunogold labeling for Arc in EVs obtained from the same Arc KO or WT cultured neuronal media in (D). Red arrow indicates a 10 nm immunogold particle (20,000×). (bottom) Quantification of EVs (vesicular structures <100 nm) that were Arc-positive±SEM, using immunogold labeling (n=3 independent experiments/EV preparations). ***p<0.001, Student's t-test.

FIG. 4 shows Arc extracellular vesicles mediate intercellular transfer of protein and mRNA in HEK293 cells. (A) Donor HEK cells in 10-cm dishes were transfected with GFP-Arc, myc-Arc, or nuclear GFP (nucGFP) for 6 h. Culture media containing plasmid DNA and transfection reagents was then removed and replaced with fresh culture media. 18 h later, this media was removed and used to replace media on naïve recipient HEK cells on coverslips in 12-well plates. 24 h later, these cells were fixed and combined FISH for Arc mRNA and ICC for Arc protein was performed. (left) Representative images of HEK cells grown on coverslips and transfected with the same protocol as in 10-cm dishes, showing Arc protein (ICC) and Arc mRNA (FISH). (Right) Representative images of recipient HEK cells showing Arc mRNA and protein were present in cells that received media from GFP-Arc- and myc-Arc-transfected cells, but not nucGFP-transfected cells. Scale bar=20 μm. Representative of 7 independent experiments and cultures. (B) Donor HEK cells in 10-cm dishes were transfected as in (A) with membrane GFP (mGFP), myc-Arc, or both constructs together. The media was replaced after 6 h, and 18 h later, transferred to naïve recipient HEK cells in 12-well plates. 24 h later, cells were fixed and combined FISH/ICC for GFP mRNA and Arc protein was performed. (left) Representative images of transfected HEK cells grown on coverslips, showing mGFP fluorescence, Arc protein and GFP mRNA. (right) Representative images of recipient HEK cells that show co-transfer of GFP protein and mRNA with Arc protein. No GFP transfer was observed in the mGFP only group. Scale bar=20 μm. Representative of 3 independent experiments and cultures.

FIGS. 5A-5D shows that Arc capsids transfer Arc mRNA into neurons. (A) Representative images of Arc ICC from DIV15 cultured hippocampal Arc KO neurons treated for 1 or 4 hr with 4 mg prArc, or WT control neurons. prArc-treated neurons show increased dendritic Arc levels relative to untreated KO neurons. (B) Neurons were treated like in (A); representative images of Arc mRNA (FISH) are shown. 4 hr of prArc treatment significantly increased dendritic Arc mRNA levels in KO neurons. (C) Representative images of Arc ICC from DIV15 cultured hippocampal KO neurons treated with 4 mg prArc, prArc-ΔCTD, or CA-prArc for 4 hr. KO neurons treated with prArc-ΔCTD and CA-prArc showed lower levels of Arc protein than prArc-treated neurons. (D) Neurons were treated like in (C); representative images of Arc mRNA are shown. Neurons treated with prArc-ΔCTD and CA-prArc showed lower levels of Arc mRNA than prArc-treated neurons. Dendritic segments boxed in white are magnified beneath each corresponding image. 30-mm segments of two dendrites/neuron were analyzed for integrated density measurements in all groups (n=10 neurons). Arc mRNA and Arc protein levels were normalized to untreated KO neurons and displayed as fold change ±SEM. Student's t-test: *p<0.05, **p<0.01, and ***p<0.001. Scale bars, 10 mm. Images are false-colored with the Smart LUT from ImageJ. All data are representative of 3-7 independent experiments using different protein preparations and cultures. See also FIG. 12.

FIGS. 6A and 6B show endogenous Arc in neuronal extracellular vesicles transfers Arc mRNA into neurons. (A) Representative images of Arc ICC from DIV15 cultured hippocampal Arc KO neurons treated for 1 or 4 hr with 10 mg of the EV fraction prepared from 10-cm dishes of DIV15 high-density cortical WT or Arc KO neurons. 1 and 4 hr treatment with KO EVs did not increase dendritic Arc levels, whereas 1 and 4 hr of treatment with WT EVs significantly increased dendritic Arc protein levels. (B) Neurons were treated like in (A); representative images of Arc mRNA (FISH) are shown. 1 and 4 hr treatment with KO EV did not increase dendritic Arc mRNA levels. 1 hr treatment with WT EV did not significantly increase dendritic Arc levels, whereas 4 hr treatment increased dendritic Arc mRNA levels. 30-mm segments of two dendrites/neuron were analyzed for integrated density measurements in all groups (n=10 neurons). Arc mRNA and Arc protein levels were normalized to untreated KO neurons and displayed as fold change ±SEM. Student's t-test: *p<0.05. **p<0.01. ***p<0.001. Scale bars, 10 mm. Representative of 6 independent experiments using different EV preparations and cultures. See also FIG. 14.

FIGS. 7A and 7B show Arc capsid- and EV-transferred Arc mRNA is accessible for activity-dependent translation. A) Representative images of Arc ICC from DIV15 cultured hippocampal Arc KO neurons treated for 4 hr with 4 mg prArc. To induce translation of Arc mRNA, 30 min prior to fixation, neurons were treated with the mGluR1/5 agonist DHPG (100 mM) for 5 min, and then drugs were washed out. 1 hr prior to fixation, a subset of neurons were pretreated with cycloheximide (CHX; 180 mM) to block protein translation. prArc significantly increased dendritic Arc expression in KO neurons, and DHPG treatment further increased dendritic Arc levels, which was blocked by pretreatment with CHX. DHPG had no effect on untreated KO neurons. (B) Representative images of Arc ICC from DIV15 hippocampal Arc KO neurons treated for 4 hr with 10 mg of the EV fraction prepared from 10-cm dishes of DIV15 high-density cortical WT or Arc KO neurons. A subset of neurons was treated with DHPG and CHX like in (A). WT EVs significantly increased dendritic Arc expression in KO neurons, whereas KO EVs had no effect. DHPG treatment had no effect on dendritic Arc expression in untreated KO neurons or KO EV-treated KO neurons. However, DHPG treatment significantly increased dendritic Arc levels in WT EV-treated KO neurons, which was blocked by pretreatment with CHX. 30-mm segments of two dendrites/neuron were analyzed for integrated density measurements in all groups (n=10 neurons). Arc mRNA and Arc protein levels were normalized to untreated KO neurons and displayed as fold change ±SEM. Student's t test: *p<0.05, **p<0.01, and ***p<0.001. Scale bars, 10 mm. Representative of 3 independent experiments using different EV/protein preparations and cultures.

FIGS. 8A and 8B show alignment of primary amino acid sequences of Ty3/Gag elements and origin of dipteran Arc genes. (A) Translated genomic DNA sequences corresponding to Arc or gypsy Gag proteins were aligned using MUSCLE (www.ebi.ac.uk/Tools/msa/muscle/). Aligned sequences were shaded using the boxshade plot server (www.ch.embnet.org/software/BOX form.html), using default parameters (50% sequences sharing amino acid identity for shading). Note: the alignments only contain fragments of Arc genes, not the full-length sequences with start sites. Species included: Mm—Mus musculus—House mouse, Hs—Homo sapiens—Human, Ac—Anolis carolinensis—Carolina anole lizard, Lc—Latimeria chalumnae—West Indian Ocean coelacanth, Dr—Danio rerio—zebrafish, Cc—Cyprinus carpio—Common carp, Dm—Drosophila melanogaster—Common vinegar fly, Ds—Drosophila suzukii—Spotted—wing drosophila, Sc—Stomoxys calcitrans—Stable fly, Lh—Linepithema humile—Argentine ant Bm—Bombyx mori—Silkworm, Tc—Tribolium castaneum—Red flour beetle. (B) (left) Maximum likelihood phylogenetic analysis of Arc homologs found in Drosophilidae, Muscidae, and Tephritidae flies. Multiple copies of darc1 were observed throughout tephritid flies, and in the house fly, Musca domestica. For each sequence, the Genbank accession number is given after the abbreviated species names. Tephritidae: RZ—Rhagoletis zephyria, CC—Ceratitis capitata, BD—Bactrocera dorsalis. Muscidae: MD—Musca Domestica, SC—Stomoxys calcitrans. (right) Putative duplication history of dArc in schizophoran flies as inferred from the phylogenetic analysis in (A). Since all schizophoran flies examined possess a homolog of darc1 and darc2, the duplication of the ancestral dArc must have occurred prior to the divergence of these species (blue triangle). This ancestral duplication event was followed by multiple rounds of duplication of darc1 (green triangles) in some of the lineages: two duplication events in the common ancestor of the Tephritidae, one additional duplication in the lineage of Ceratitis capitata, and one additional duplication in the lineage of Bactrocera dorsalis. Independently, darc1 experienced three rounds of duplication in the lineage of Musca domestica. By contrast, darc2 has apparently remained a single copy gene in the species examined. The sequences on the left from top to bottom represent SEQ ID NOs:11-27. The sequences on the right from top to bottom represent SEQ ID NOs:28-45.

FIGS. 9A-9D shows recombinant protein purification and experiments relating to FIG. 2. A) (left to right) Representative Coomassie gel of affinity purifications of full-length rat Arc (prArc), prArc-ΔCTD, CA-prArc, GST, and Endo3A showing similar expression levels to that of prArc. prArc-ΔCTD and Endo3A were prepared in the same manner as prArc. GST was directly eluted from the affinity resin using 15 mML-glutathione. His-tagged CA-prArc was eluted from Ni2+ affinity resin using 250 mM imidazole. All proteins were then buffer exchanged into 150 mM NaCl, 50 mM Tris, pH 7.4 following GST-tag cleavage by Precision Protease or elution. Buffer conditions were adjusted for all proteins for each experiment: 500 mM NaPO4, 50 mM Tris, pH 7.4 for capsid stability. Analyses showing the partitioning of bacterially-expressed protein into soluble (sup) and insoluble (pellet) fractions (lanes 1, 2), capture of the protein on a GST or Ni2+ affinity matrices (lanes 3-5 show the flow through (FT), wash and captured protein, respectively). This panel demonstrates the protein expression levels and the efficacy and efficiency of affinity capture. (B) Representative Coomassie gels of peak fractions of prArc, prArc-ΔCTD, and Endo3A eluted from 5200 size exclusion columns. Peak fractions were pooled and concentrated to a final stock concentration of 1 mg/mL. prArc was concentrated to 1 mg/mL from each purification for use in all biochemistry/EM experiments, unless noted. For cell biology experiments, prArc was diluted to 0.4 mg/mL and 4 μg total protein was used per condition. (C) Representative Coomassie gel of affinity purification of Drosophila dArc1 from BL21 bacteria lysates demonstrating similar expression levels to rat prArc. (D) HEK293 cells in 12-well plates were transfected with full-length rat WT Arc or GFP plasmids using Lipofectamine at equal DNA concentrations and subjected to formaldehyde crosslinking in situ Cell lysates were blotted with anti-GFP or anti-Arc antibodies. Note that higher molecular species corresponding to Arc dimers and trimers can be observed in the crosslinked Arc sample, but not in the GFP sample.

FIGS. 10A-10D RNA binding experiments and properties of Arc EVs, related to FIGS. 2 and 3. (A) Representative Coomassie gels of nucleotide stripping of prArc. (left) Cells were lysed in 20 mM NaCl, 50 mM Tris, 2 mM MgCl2, 5% glycerol, 1 mM DTT, pH 8.0. Fractions shown are supernatant and pellet fractions of cellular lysis after pelleting at 21,000×g for 45 min. The supernatant from this step was treated with 0.1% PEI to precipitate nucleic acids. This treatment resulted in a shift in the A260/280 ratio from 1.71±0.018 to 1.29±0.023, indicating a drop in nucleic acid content. The sample was pelleted at 27,000×g for 20 min and the resulting supernatant was treated with ammonium sulfate (AmSulf) precipitation to concentrate Arc and pelleted at 10,000×g for 10 min. The AmSulf pellet containing Arc was then subjected to affinity purification as above. (right) Representative Coomassie gel of peak fractions of cleaved, affinity purified PEI treated Arc from an anion exchange column. This chromatography step further stripped bound nucleic acids from Arc. Peak fractions were concentrated to 1 mg/mL and the final measured A260/280 ratio for these fractions was 0.68±0.03 (n=3), indicating that PEI-treated prArc was largely free of nucleic acids. (B) (left) Representative negative stain EM images of purified EVs from Arc-transfected HEK293 cell media collected for 24 h used for western blot analysis. (right) Representative negative stain EM images of purified EVs from WT cultured neuron media collected for 24 h used for western blot analysis. Red arrows indicate purified EVs. (C) (left) Western blot of Arc in untreated EVs or EVs treated with trypsin (0.05 mg/mL) for 30 min. prArc was used as a positive control for trypsin activity. (right) Quantification of Arc western blot normalized to total protein. Trypsin degraded prArc but had no effect on Arc protein present in neuronal EVs. (D) Activity dependence of Arc secretion. Purified EV fraction from media collected from DIV15 cortical neurons in 10-cm dishes from untreated WT neurons was compared with treatment with KCl. Media was freshly exchanged with basal media or media supplemented with KCl to a final concentration of 50 mM. Following media exchange, cells were incubated for 1 h and media was collected and the EV fraction was purified. (left) Western blots of Arc and total protein from the purified EV fraction from cultured neuronal media. (right) Quantification of Arc protein levels, normalized to total protein. KCl treatment resulted insignificantly more Arc released into the media (n=2; p<0.05).

FIGS. 11A-11C shows HEK cell experiments and custom-made Arc antibody control experiments, related to FIG. 4. (A) HEK293 cells in 10-cm dishes were transfected with GFP-Arc as in FIG. 4. 18 h later, media from GFP-Arc-transfected HEK cells in 10-cm dishes was transferred to naïve HEK cells in 12-well plates, and 80 mM Dynasore was added in one group at the same time to block endocytosis. After 6 h, the Dynasore-treated media was exchanged for fresh HEK media. 18 h later, cells were fixed and clusters of GFP-Arc-expressing cells over an entire 18 mm coverslip were manually counted through a 20× objective (n=3 coverslips/group). (left) Representative images of one 20× field of view. (right) Dynasore significantly reduced the number of clusters of GFP-Arc-positive cells over the entire coverslip. Student's t-test: *p<0.05. Scale bar=50 mm. Representative of three independent experiments and cultures with similar results. (B) DIV15 cultured hippocampal Arc KO and WT neurons were immunostained for the dendritic protein MAP2 with Alexa Fluor 555 only (top row), or both MAP2 (Alexa 555) and Arc (Alexa 488; bottom two rows). Imaging settings for Arc were determined based on Arc immunostaining in WT neurons (bottom row). No GFP fluorescence from GFP knocked in to the Arc locus in the KO neurons was visible under these imaging conditions. Example of two independent experiments. (C) DIV15 cultured hippocampal Arc KO and WT neurons were fixed and immunostained with either a custom-made rabbit polyclonal Arc antibody (ProteinTech) or the commercially available Synaptic Systems rabbit polyclonal Arc antibody, as well as the dendritic protein MAP2. All groups were imaged with the same acquisition settings. 30-mm segments of two dendrites/neuron were analyzed in all groups (n=10 neurons) and were chosen using MAP2 staining. Both antibodies were able to detect a difference between Arc KO and WT neurons, although the signal:noise was better using the custom-made antibody. Arc in the soma/nucleus varies widely from neuron to neuron with both antibodies, under basal conditions. Student's t-test: **Arc KO versus WT with custom antibody, p<0.01. # Arc KO versus WT with Synaptic Systems (“SySy”) antibody, p<0.05. Arc images are false-colored with the Smart LUT in ImageJ to better display differences in Arc expression. Example of two independent experiments.

FIGS. 12A-12D show experiments relating to FIG. 5. (A) To test whether Arc mRNA is protected in prArc capsids, samples were subjected to 15 min treatment with RNase A, then RNase inhibitor (1 U/mL) to quench activity, prior to incubation with neurons. (left) Representative images of Arc mRNA in DIV15 cultured hippocampal Arc KO neurons incubated with the treated or untreated prArc samples for 4 h. (right) prArc treatment resulted an increase in dendritic Arc mRNA levels in Arc KO neurons. prArc treated with RNase did not affect Arc mRNA transfer. (B) DIV15 cultured hippocampal Arc KO neurons were treated for 4 h with 4 mg prArc. In one group, 30 min before prArc was added, neurons were pretreated with 80 m M Dynasore to block endocytosis. (left) Representative images of Arc protein and mRNA levels. (right) Pretreatment with Dynasore significantly blocked uptake/transfer of prArc protein and Arc mRNA. Student's t-test: *p<0.05. ***p<0.001. Example of three independent experiments (A, B). Scale bars in all panels=10 mm. (C) DIV15 cultured hippocampal Arc KO neurons were treated for 4 h with 4 mg prArc. Either combined FISH/ICC for Arc mRNA and Rab5 protein, or ICC for Arc and Rab5 protein, was performed. (left) Representative images of dendrites showing Arc mRNA plus Rab5 protein or Arc and Rab5 protein. (right) Arc protein and mRNA showed around 50% colocalization in dendrites with Rab5. White arrowheads indicate Arc alone, and yellow arrowheads indicate Arc/Rab5 colocalization. Example of two independent experiments. Scale bar=10 mm. (D) Purified protein samples of prArc, prArc(RNA−), prArc-ΔCTD, and CA-prArc were separated by SDS-PAGE, and the resulting western blot was immunostained for Arc using our custom-made Arc antibody. The antibody successfully detected all of the mutant constructs, suggesting that the lack of Arc immunostaining observed in transfer experiments was not a result of an inability of the antibody to detect the mutants. “Total” is Ponceau stain for total protein for each sample.

FIG. 13 shows purified Arc stripped of nucleic acids binds the outside of neurons and is not internalized. DIV15 cultured hippocampal Arc KO neurons were treated with 4 mg prArc or prArc(RNA−) for 4 h before being fixed. One group from each treatment was not permeabilized during the immunocytochemistry procedure for Arc and MAP2. prArc-treated neurons that were non-permeabilized showed little to no MAP2 and Arc immunostaining. However, prArc(RNA−)-treated neurons showed no difference in Arc immunostaining between permeabilized and non-permeabilized conditions, although MAP2 immunostaining was still absent in the non-permeabilized condition, suggesting that prArc(RNA−) accumulates on the outside of the neurons. Dendritic segments boxed in white are shown magnified beneath each corresponding image. Scale bars=10 mm. Example of three independent experiments. Arc images are false-colored with the Smart LUT from ImageJ to highlight differences in Arc expression. Merged images have MAP2 immunostaining in magenta and Arc in green.

FIGS. 14A, 14B, 14C, and 14D show RNase and Uptake experiments, related to FIG. 6. (A) To test whether Arc mRNA is protected in neuronal EVs, EVs prepared from 10-cm dishes of DIV15 cultured WT cortical neurons were subjected to 15 min treatment with RNase A, then RNase inhibitor (1 U/mL) to quench activity, prior to incubation with neurons. DIV15 cultured hippocampal Arc KO neurons were incubated with 10 mg of the treated or untreated WT EV samples for 4 h. (left) Representative images of Arc mRNA levels in neurons. (right) WT EV treatment resulted an increase in dendritic Arc mRNA levels in Arc KO neurons. WT EV treated with RNase did not affect Arc mRNA transfer. (B) DIV15 cultured hippocampal Arc KO neurons were treated for 4 h with 10 mg of the EV fraction harvested from the media of 10-cm dishes containing DIV15 high-density cultured cortical WT or Arc KO neurons. In one group, 30 min before EVs were added, neurons were pretreated with 80 mM Dynasore to block endocytosis. (top) Representative images of Arc protein levels (left) or Arc mRNA levels (right). (bottom) Pretreatment with Dynasore significantly blocked uptake of Arc protein and mRNA from WT EVs. Arc protein and mRNA expression was normalized to Arc KO and is displayed as fold-change ±SEM. Dendritic segments boxed in white are shown magnified beneath each corresponding image. 30-mm segments of two dendrites/neuron were analyzed in all groups (n=10 neurons) and were chosen using MAP2 staining. Student's t-test: ***p<0.001. Scale bars=10 mm. Example of three independent experiments. Images are false-colored with the Smart LUT from ImageJ to highlight differences in Arc expression. (C) DIV15 cultured hippocampal Arc KO neurons were treated for 4 h with 10 mg of WT EVs, then fixed. Either combined FISH/ICC for Arc mRNA and Rab5 protein, or ICC for Arc and Rab5 protein, was performed. (left) Representative images of dendrites showing Arc mRNA plus Rab5 protein or Arc and Rab5 protein. (right) Arc mRNA and protein showed 30%-40% colocalization in dendrites with Rab5. White arrowheads indicate Arc alone, and yellow arrowheads indicate Arc/Rab5 colocalization. Example of two independent experiments. Scale bar=10 mm. (D) Model: Comparison of HIV Gag and Arc capsid life cycle. (top) HIV Gag protein self-assembles (via the CA domain) in the cytosol and at the plasma membrane (by myristoylation of the MA domain), while the capsid encapsulates viral RNA (via the NC domain). The immature HIV capsid is released from the cell in an ESCRT-dependent manner (via the p6 domain) with membrane that contains the viral envelope protein (Env). The mature virus particles bind host cells through surface receptors (such as CD4) and membrane fusion occurs. Alternatively, in some cell types virus particles are first endocytosed prior to fusion and particles released into the cell after full fusion occurs in the endosome. Viral RNA is released and then reversed transcribed into viral DNA that is integrated into the host genome. (bottom) Arc mRNA is trafficked out into dendrites in RNA granules that contain a selection of different mRNAs. Local translation of Arc mRNA takes place in dendrites in response to neuronal activity. High concentrations of Arc protein self-assemble and form Arc capsids, which encapsulate select mRNAs that are spatially proximal, including Arc mRNA. Arc capsids are released from dendrites in Arc Capsids Bearing Any RNA (ACBARs) and transfer of mRNA and other putative cargo takes place in neighboring dendrites.

FIGS. 15A and 15B show that RNA co-transferred with Arc protein is translated in recipient cells. (A) HEK293T cells “donor” cells were co-transfected with WT myc-Arc and GFP. Media from transfected cells was placed on naïve, “recipient” cells with or without the translation inhibitor cycloheximide (CHX). 6 h later, cells were fixed and fluorescent in situ hybridization performed for GFP RNA and immunocytochemistry performed for Arc protein. (B) CHX treatment significantly reduced the amount of GFP protein expressed in recipient cells, without affecting GFP RNA levels, as shown by a shift to the left in the cumulative frequency distribution and a reduction in average GFP and Arc protein levels per cell. This indicates that Arc protein co-transfers GFP RNA that can be newly translated in recipient cells. *p<0.05. **p<0.01. ***p<0.001. Scale bar=10 μm.

Additional advantages of the disclosed method and compositions will be set forth in part in the description which follows, and in part will be understood from the description, or may be learned by practice of the disclosed method and compositions. The advantages of the disclosed method and compositions will be realized and attained by means of the elements and combinations particularly pointed out in the appended claims. It is to be understood that both the foregoing general description and the following detailed description are exemplary and explanatory only and are not restrictive of the invention as claimed.

### DETAILED DESCRIPTION

The disclosed method and compositions may be understood more readily by reference to the following detailed description of particular embodiments and the Example included therein and to the Figures and their previous and following description.

It is to be understood that the disclosed method and compositions are not limited to specific synthetic methods, specific analytical techniques, or to particular reagents unless otherwise specified, and, as such, may vary. It is also to be understood that the terminology used herein is for the purpose of describing particular embodiments only and is not intended to be limiting.

Disclosed are materials, compositions, and components that can be used for, can be used in conjunction with, can be used in preparation for, or are products of the disclosed method and compositions. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a nucleic acid sequence capable of encoding an Arc protein is disclosed and discussed and a number of modifications that can be made to a number of molecules including the nucleic acid sequence are discussed, each and every combination and permutation of the nucleic acid sequence and the modifications that are possible are specifically contemplated unless specifically indicated to the contrary. Thus, if a class of molecules A, B, and C are disclosed as well as a class of molecules D, E, and F and an example of a combination molecule, A-D is disclosed, then even if each is not individually recited, each is individually and collectively contemplated. Thus, is this example, each of the combinations A-E, A-F, B-D, B-E, B—F, C-D, C-E, and C—F are specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B-F, and C-E are specifically contemplated and should be considered disclosed from disclosure of A, B, and C; D, E, and F; and the example combination A-D. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods, and that each such combination is specifically contemplated and should be considered disclosed.

### A. Definitions

It is understood that the disclosed method and compositions are not limited to the particular methodology, protocols, and reagents described as these may vary. It is also to be understood that the terminology used herein is for the purpose of describing particular embodiments only, and is not intended to limit the scope of the present invention which will be limited only by the appended claims.

It must be noted that as used herein and in the appended claims, the singular forms “a”, “an”, and “the” include plural reference unless the context clearly dictates otherwise. Thus, for example, reference to “an Arc capsid” includes a plurality of such Arc capsids, reference to “the Arc capsid” is a reference to one or more Arc capsids and equivalents thereof known to those skilled in the art, and so forth.

“Optional” or “optionally” means that the subsequently described event, circumstance, or material may or may not occur or be present, and that the description includes instances where the event, circumstance, or material occurs or is present and instances where it does not occur or is not present.

Ranges may be expressed herein as from “about” one particular value, and/or to “about” another particular value. When such a range is expressed, also specifically contemplated and considered disclosed is the range from the one particular value and/or to the other particular value unless the context specifically indicates otherwise. Similarly, when values are expressed as approximations, by use of the antecedent “about,” it will be understood that the particular value forms another, specifically contemplated embodiment that should be considered disclosed unless the context specifically indicates otherwise. It will be further understood that the endpoints of each of the ranges are significant both in relation to the other endpoint, and independently of the other endpoint unless the context specifically indicates otherwise. Finally, it should be understood that all of the individual values and sub-ranges of values contained within an explicitly disclosed range are also specifically contemplated and should be considered disclosed unless the context specifically indicates otherwise. The foregoing applies regardless of whether in particular cases some or all of these embodiments are explicitly disclosed.

Unless defined otherwise, all technical and scientific terms used herein have the same meanings as commonly understood by one of skill in the art to which the disclosed method and compositions belong. Although any methods and materials similar or equivalent to those described herein can be used in the practice or testing of the present method and compositions, the particularly useful methods, devices, and materials are as described. Publications cited herein and the material for which they are cited are hereby specifically incorporated by reference. Nothing herein is to be construed as an admission that the present invention is not entitled to antedate such disclosure by virtue of prior invention. No admission is made that any reference constitutes prior art. The discussion of references states what their authors assert, and applicants reserve the right to challenge the accuracy and pertinency of the cited documents. It will be clearly understood that, although a number of publications are referred to herein, such reference does not constitute an admission that any of these documents forms part of the common general knowledge in the art.

Throughout the description and claims of this specification, the word “comprise” and variations of the word, such as “comprising” and “comprises,” means “including but not limited to,” and is not intended to exclude, for example, other additives, components, integers or steps. In particular, in methods stated as comprising one or more steps or operations it is specifically contemplated that each step comprises what is listed (unless that step includes a limiting term such as “consisting of”), meaning that each step is not intended to exclude, for example, other additives, components, integers or steps that are not listed in the step.

As used herein, the term “mutation” includes the addition, deletion, or substitution of an amino acid or nucleic acid.

### B. Arc Capsid

Disclosed are Arc capsids. The Arc capsids can be comprised of one or more Arc proteins. The one or more Arc proteins can be all from the same species or from one or more species. In some aspects, Arc capsids can include recombinant Arc capsids comprising Arc proteins from two or more species. The disclosed Arc capsids are recombinant, in that they are not naturally occurring. Recombinant Arc capsids can include an Arc capsid comprising Arc proteins from two or more species or can comprise an Arc capsid carrying a nucleic acid sequence not naturally found in an Arc capsid. In some aspects, the Arc capsids disclosed herein can comprise a combination of naturally occurring and non-naturally occurring Arc proteins. For example, the Arc capsid can comprise a naturally occurring Arc protein and a recombinant, non-naturally occurring Arc protein sequence. In some aspects, Arc capsids can comprise 1-50, 1-100, 1-150, 1-200, 1-250, 1-300, 1-350, 1-400, 1-450, 1-500, 1-550, 1-600, 1-650, 1-700, 1-750, 1-800, 1-850, 1-900, 1-950 or 1-1000 Arc proteins.

Disclosed are Arc capsids conjugated to a labeling moiety. A labeling moiety can be, but is not limited to, fluorescent molecules, phosphorescent molecules, enyzmes, antibodies, ligands, proteins, and radioactive isotopes. Examples of labeling moieties include, but are not limited to, GFP, myc, XFP, HALO, His, RFP, biotin, and FITC. In some aspects, labeling moieties can be used for detecting the Arc capsids. In some aspects, labeling moieties can be used for purifying Arc capsids. In some aspects, labeling moieties can be used to target specific protein interactions.

Disclosed are Arc capsids conjugated to a targeting moiety. A targeting moiety refers to the portion of the conjugate that specifically binds to a selected target. The targeting moiety can be, for example, a polysaccharide, a peptide, peptide ligand, an oligonucleotide, an aptamer, an antibody or fragment thereof, a single chain variable fragment (scFv) of an antibody, or a Fab fragment, or a nanobody. As used herein, a “targeting moiety” can be specific to a recognition molecule on the surface of a cell or a population of cells, such as, for example B cells, T cells, or neurons. Thus, disclosed are Arc capsids conjugated to a targeting moiety, wherein the targeting moiety is a cell-specific targeting moiety.

Disclosed are Arc capsids conjugated to a targeting moiety, further comprising a labeling moiety.

In some aspects, the nucleic acid sequence carried by the Arc capsid can be DNA or RNA. In some aspects, the DNA can be single stranded or double stranded. In some aspects, the RNA sequence can be, but is not limited to, mRNA, RNAi, or micro RNA.

Disclosed are Arc capsids comprising a heterologous nucleic acid sequence. For example, a heterologous nucleic acid sequence can be any nucleic acid sequence that is not derived from the same cell as the Arc capsid. In some aspects, the heterologous nucleic acid sequence is a non-Arc mRNA sequence.

In some aspects, the Arc capsids can carry a nucleic sequence that can be transferred from the Arc capsid to a cell. In some aspects, a transferred mRNA sequence can be translated once inside a cell.

In some aspects, the disclosed Arc capsids can be mammalian. In some aspects, the Arc capsids can be drosophila derived Arc capsids. In some aspects, the Arc capsid can be an Arc capsid homologue. In some aspects, the Arc capsid homologue can be from any species.

In some aspects, the disclosed Arc capsids can be 10-200 nm. In some aspects, the disclosed Arc capsids can be 10-80 nm. In some aspects, the disclosed Arc capsids can be 30-40 nm. In some aspects, the disclosed Arc capsids can be 10-100 nm. In some aspects, the disclosed Arc capsids can be 100-200 nm.

1. Arc Proteins

Disclosed are Arc proteins comprising the amino acid sequence of any known Arc proteins. In some aspects, the amino acid sequence can be the amino acid sequence of SEQ ID NO:1, rat Arc protein:

(SEQ ID NO: 1)
meldhmttgg lhaypaprgg paakpnvilq igkcraemle
hvrrthrhll tevskqvere lkglhrsvgk lennldgyvp
tgdsqrwkks ikacicrcqe tianlerwvk remhvwrevf
yrlerwadrl esmggkypvg separhtvsv gvggpepycq
eadgydytvs pyaitpppaa gelpeqesvg aqqyqswypg
edgqpspgld tqifedpref lshleeylrq vggseeywls
qiqnhmngpa kkwwefkqgs vknwyefkke flqysegtls
reaigreldl pqkqgepldq flwrkrdlyq tlyvdaeeee
iiqyvvgtlq pkfkrflrhp lpktleqliq rgmevqdgle
qaaepsvtpl ptedetealt paltsesvas drtqpe

In some aspects, the amino acid sequence can be the amino acid sequence of SEQ ID NO:2, human Arc protein:

(SEQ ID NO: 2)
meldhrtsgg lhaypgprgg qvakpnvilq igkcraemle
hvrrthrhll aevskqvere lkglhrsvgk lesnldgyvp
tsdsqrwkks ikaclcrcqe tianlerwvk remhvwrevf
yrlerwadrl estggkypvg sesarhtvsv gvggpesych
eadgydytvs pyaitpppaa gelpgqepae aqqyqpwvpg
edgqpspgvd tqifedpref lshleeylrq vggseeywls
qiqnhmngpa kkwwefkqgs vknwvefkke flqysegtls
reaigreldl pqkqgepldq flwrkrdlyq tlyvdadeee
iiqyvvgtlq pklkrflrhp lpktleqliq rgmevqddle
qaaepagphl pvedeaetlt papnsesvas drtqpe

Disclosed are Arc proteins comprising at least one mutation in the CA domain (amino acids 207-370). In some aspects, disclosed are Arc proteins comprising at least one mutations in the C-terminal domain (amino acids 278-370) of the CA domain. Disclosed are Arc proteins comprising at least one mutation in amino acids 278-370 of SEQ ID NO:1 or SEQ ID NO:2. Disclosed are Arc proteins comprising at least one mutation in an amino acid that corresponds to amino acids 278-370 of SEQ ID NO:1 or SEQ ID NO:2. In some aspects, disclosed herein are Arc proteins that comprise a deletion of amino acids 278-370 of the CA domain (the CA domain comprises amino acids 207-370 of SEQ ID NOS 1 or 2).

Disclosed are Arc proteins comprising at least 60, 65, 70, 75, 80, 85, 90, 95, or 99.9% identity to any of the known or disclosed Arc amino acid sequences. For example, disclosed are Arc proteins proteins comprising at least 60, 65, 70, 75, 80, 85, 90, 95, or 99.9% identity to SEQ ID NO:1. Also disclosed are Arc proteins proteins comprising at least 60, 65, 70, 75, 80, 85, 90, 95, or 99.9% identity to SEQ ID NO:2.

2. Arc Nucleic Acids

Disclosed are nucleic acid sequences capable of encoding any known Arc protein.

Disclosed are nucleic acid sequences capable of encoding an Arc protein comprising the sequence of SEQ ID NO:1. Disclosed are nucleic acid sequences capable of encoding an Arc protein comprising the sequence of SEQ ID NO:2. For example, disclosed are nucleic acid sequences comprising the sequence of SEQ ID NO:3, the nucleic acid sequence for the rat Arc gene.

(SEQ ID NO: 3)
agtgctctgg cgagtagtcc tccctcagcc gcagtctctg ggcctcttca gcttgagcgg cggcgagcct gccacactcg
ctaagctcct ccggcaccgc gcacttgcca ctgccactgc cgcttcgcgc ccgctgcagc cgccggctct gaatccttct
ggcttccgcc tcagaggagt tcttagcctg tcccgaaccg taaccccggc gagcagatgg agctggacca tatgacgacc
ggcggcctcc acgcctaccc tgccccgcgg ggtgggccgg ccgccaaacc caatgtgatc ctgcagattg gtaagtgccg
agctgagatg ctggagcacg tacggaggac ccaccggcat ctgttgaccg aagtgtccaa gcaggtggag cgagagctga
aagggttgca caggtcggtg ggcaagctgg agaacaactt ggacggctat gtgcccacgg gcgactcaca gcgctggaag
aagtccatca aggcctgtct ctgccgctgc caggagacca tcgccaacct ggagcgctgg gtcaagcgtg agatgcacgt
gtggagggag gtcttctacc gtctggagag gtgggccgac cgcctggagt ccatgggcgg caagtaccca gtgggcagcg
agccggcccg ccacactgtc tctgtaggtg tggggggtcc agagccctac tgccaggaag ctgatggcta cgactacact
gttagcccct atgccatcac cccgccacct gccgcaggag agctgcctga gcaggagtca gttggggctc agcaatacca
gtcttgggtg ccaggtgagg atgggcaacc aagcccaggt ctggataccc agatctttga ggacccacgg gagttcctga
gccacctgga agagtacctg cggcaggtgg gtggctctga agaatattgg ctgtcccaga tccagaacca catgaatggg
ccagccaaga agtggtggga gttcaaacag ggctcggtga agaactgggt ggagttcaag aaggagtttc tgcagtacag
tgagggtacg ctctcccgcg aagccattca gcgggagctg gacctgccac agaagcaggg tgagccactt gaccagttcc
tctggcgtaa gcgggacctg taccagacac tgtatgtgga cgctgaggag gaggagatca ttcagtatgt ggtgggcacc
ctgcagccca agttcaagcg ctttctgcgc cacccacttc ccaagaccct ggagcagctc atccagaggg gcatggaagt
tcaggacggc ctggagcagg cagctgagcc ttctgtcacc cctctgccca cagaggatga gactgaggca ctcacgcctg
ctcttaccag cgagtcagta gccagtgaca ggacccagcc tgaatagagg ggccagccca gggtccccag cctgcctgcc
acacccagtc tgtggctttt gtcaactagg acttgattga gctggggctg acacccaagg ggatgccctg tccagccaga
caccttctca cccactggcc tgactcacaa ctgccacaca accatgattc atggacatca agaagcccct ctcccatagg
gctcccacct gccacctacc cctcacctgt ctgccctagt cctggccctg tctccagtgg cctcaccctc tacactctca
gaccatcaca gaacaccttt ggcttcctca ttctgcatca gtgtccaggg ccctttgggt agtcaagaaa tcaagtgtct
gaaaggcaat gaaaagtagg caccaaaccc aaggggcatc ccagggcaga tgctaaagca gaatcagaga tggccgaagg
aacctctact tccggggatg cagcccgctc ctacagacac agcagatcca gctggtgccc tacctgcctc ccagagcaac
tggccagtct tgggcagcat agctcccctc tcagggtgag ctgaagcagc agacctgacg cgctggcgcc tcctggcccc
cagcagtgat tcataccagt gaagaaaagc agacttcggc tccatgactc agccatgcca ggcggagggt cccagagggg
ctgagtcctc agccccagct gaggcagcag ctggagtctt cagagccagg tgaatgacac caggtctcaa gctgctgaga
agtctttccg gccatgtctg gaaggggtac caccccagca ccagcaccgt cccctcctct cttgaagctg cctgcacaga
ggttccaaga cactttcaag gcagagaaaa taggattaca aagaggaggt gccttggcag agggcagcac ccagctcagc
ctcagagctg aaggtgaaga caagccagcg tgaaaccccg ggtctgccac gaatgcccgc tccgctggcc actcaccagc
tgcctgccac aagccactgc agcttgagca gggtctgtgc cctctcagca cagagcccag ttcgctgcgt ggcctttggc
ccccgccaga accttgcagg agccttaagg ttcgggccct agcccagcct gaccttacct gctgtgccct gcctgctggt
caagtccagt cccaggagac cccatgcctt ggctcctagg ctgttccagg cacttccctg acctgccggg tgattgccca
gctggaacct catccacacc ccagcaccaa ccacctcgtg ttggtaactg ctcgtgtctg tagtctgagt aggccatgtt
gaggttcctc catctgcctg gtccattggt gttctgagac cagttccact gctgttctga cagatccccc accctgtgcc
cctgccagcc cccacaggtt tatttttgca cataaaccat gacccatact aatttggcta gctctgggga ctagggagac
cctggagatc tcaagagtgt ggctatcccc tattttcacc aagccttcaa tatccagcca ggccatctgg cccacaccat
cttacctcaa agacagacat atatatatat atacatatat atgattttgt taataaaact atgaaattta aa

Also disclosed are nucleic acid sequences comprising the sequence of SEQ ID NO:4, the nucleic acid sequence for the human Arc gene.

(SEQ ID NO: 4)
cgcgtgggcc gcagcagccg agccggacct gcctccccgg gcgtgctccg ccggccccgc cgccggcccg cagcgacaga
caggcgctcc ccgcagctcc gcacgggacc caggccgccg gaccccagcg ccggaccacc gtccgtccgc cccgaggagt
ttgccgcctg ccggagcacc tgcgcacaga tggagctgga ccaccggacc agcggcgggc tccacgccta ccccgggccg
cggggcgggc aggtggccaa gcccaacgtg atcctgcaga tcgggaagtg ccgggccgag atgctggagc acgtgcggcg
gacgcaccgg cacctgctgg ccgaggtgtc caagcaggtg gagcgcgagc tgaaggggct gcaccggtcg gtcgggaagc
tggagagcaa cctggacggc tacgtgccca cgagcgactc gcagcgctgg aagaagtcca tcaaggcctg cctgtgccgc
tgccaggaga ccatcgccaa cctggagcgc tgggtcaagc gcgagatgca cgtgtggcgc gaggtgttct accgcctgga
gcgctgggcc gaccgcctgg agtccacggg cggcaagtac ccggtgggca gcgagtcagc ccgccacacc gtttccgtgg
gcgtgggggg tcccgagagc tactgccacg aggcagatgg ctacgactac accgtcagcc cctacgccat caccccgccc
ccagccgctg gcgagctgcc cgggcaggag cccgccgagg cccagcagta ccagccgtgg gtccccggcg aggacgggca
gcccagcccc ggcgtggaca cgcagatctt cgaggaccct cgagagttcc tgagccacct agaggagtac ttgcggcagg
tgggcggctc tgaggagtac tggctgtccc agatccagaa tcacatgaac gggccggcca agaagtggtg ggagttcaag
cagggctccg tgaagaactg ggtggagttc aagaaggagt tcctgcagta cagcgagggc acgctgtccc gagaggccat
ccagcgcgag ctggacctgc cgcagaagca gggcgagccg ctggaccagt tcctgtggcg caagcgggac ctgtaccaga
cgctctacgt ggacgcggac gaggaggaga tcatccagta cgtggtgggc accctgcagc ccaagctcaa gcgtttcctg
cgccaccccc tgcccaagac cctggagcag ctcatccaga ggggcatgga ggtgcaggat gacctggagc aggcggccga
gccggccggc ccccacctcc cggtggagga tgaggcggag accctcacgc ccgcccccaa cagcgagtcc gtggccagtg
accggaccca gcccgagtag agggcatccc ggagccccca gcctgcccac tacatccagc ctgtggcttt gcccaccagg
acttttgagc tggggctgac tcctgcaggg gaagccctgg tccagctggg tgccccctcg agctccgggc ggactcgcac
acactcgtgt catccagatg tgagcaccgc acccagcggc aaagagccct cccccctgca gggctccacc catcaccctc
cctccgtctg tctttccggc ctggacccca ccctccacac tctcaggcca tcacagaaca ccccagcttc ctcattctgc
tacaacaccc aggccctctg gacatccaga aaaccaagtg tccggatggc aggggccagc ggccaccaag ctcatgggac
acccagagca gaagctaggg cagagccaat gctgagggag cctcgacttc cggcgccgcc gccctctccc ggcatccgca
gagccagctg acgccctccc tgcctcccag ggcagctggc cagcctcggg cagcgcggcc ccctcctccc aggggagagt
agaagtcgca cacgcagcag agcagacctg atgtcccggt gcttcctggc ccctcagctc cagtgattca cgcccgcctg
gagaagaatc agagctcagc tcatgactca cccatggcag gcggagggtc ccagaggggc tgagtcctca aatccggctg
aggcagcagc tggcaccatc agagccagga gagtgacaac aggtctcaag gttcccacaa agtctttgct gctgtgctgg
gcaccaccca cccctcacct tgcaggctgc ctgcgtggga ggcgaagtcc caggacagcc cagagggggg ctacagagag
gagtcggctg cagcagaggg caggagcccc agcttagccc tgagcgccag cgcgaggacc agggcctgcc actaagcccg
ccccgctggc cgccagctgc ccgtccccag agccactgca gcaggagtcg ggccctgcct ccctcccagc agggaaaccc
cgcccgctgc caggccatcc tctctgccag aggctttcat gagccccaag gctggggcca cagctcctac ccctgcccag
cagccctgag ctcagctgca ggaaggacat cccagaagcc atggctcctg gggcgcttcc aggcattctg ccctgccccg
acaccagaac cctggtgctg gtgggccact agcgtctgca gcctaagcag gtgctggctc agggttcatc attctgcctt
gtccactggg ggaccagccc tgcagaccac tctgacaagt cttcagccca caccttgcca gccccacaga ttttattttt
gcacataagc cataaccaat cctcaaggct ggcacaggct ttggggaagc cctggagcct gtgaagaccc tggaaacctc
atgaggctgt ggccaacccc tgccccttgc cccacacaga ccaggcctta aatgtcggtc caggccctgt gcaccttacc
ccagagacag actctttttg taagattttg ttaataaaac actgaaactt c

Disclosed are nucleic acid sequences comprising at least 60, 65, 70, 75, 80, 85, 90, 95, or 99.9% sequence identity to SEQ ID NO:3. Disclosed are nucleic acid sequences comprising at least 60, 65, 70, 75, 80, 85, 90, 95, or 99.9% sequence identity to SEQ ID NO:4.

Disclosed are nucleic acid sequences comprising at least one mutation in a sequence that is capable of encoding amino acids 278-370 of SEQ ID NO:1 or SEQ ID NO:2. In other words, disclosed are nucleic acid sequences comprising at least one mutation in nucleic acids 832-1110 of SEQ ID NO:3 or SEQ ID NO:4. Also disclosed are nucleic acid sequences comprising at least one mutation in a sequence that is capable of encoding amino acids 207-370 of SEQ ID NO:1 or SEQ ID NO:2. In other words, disclosed are nucleic acid sequences comprising at least one mutation in nucleic acids 619-1110 of SEQ ID NO:3 or SEQ ID NO:4.

Also disclosed are nucleic acid sequences capable of encoding a protein that shares secondary or tertiary structure to an Arc protein described herein.

### C. Vectors

Disclosed are vectors comprising a nucleic acid sequence capable of encoding an Arc protein. In some aspects, the Arc protein can be any of the Arc proteins disclosed herein.

Disclosed are vectors comprising a nucleic acid sequence capable of encoding a protein that shares secondary or tertiary structure to an Arc protein described herein.

In some aspects, the disclosed vectors can further comprise a nucleic acid sequence capable of encoding a labeling moiety. In some aspects, the labeling moiety can be any peptide or protein that is encoded for by a nucleic acid. For example, the labeling moiety can be, but is not limited to, GST, myc, His, or GFP.

In some aspects, the labeling moiety can be operably linked to the nucleic acid sequence capable of encoding the Arc protein. Thus, the labeling moiety and the Arc protein can be transcribed together.

In some aspects, the disclosed vectors can further comprise a nucleic acid sequence capable of encoding a targeting moiety. In some aspects, the targeting moiety can be operably linked to the nucleic acid sequence capable of encoding the Arc protein. Thus, the targeting moiety and the Arc protein can be transcribed together. In some aspects, targeting moiety can be, but is not limited to, a polysaccharide, a peptide, peptide ligand, an oligonucleotide, an aptamer, an antibody or fragment thereof, a single chain variable fragment (scFv) of an antibody, or a Fab fragment, or a nanobody.

In addition to a nucleic acid sequence capable of encoding an Arc protein, the disclosed vectors can carry regulatory sequences that control the expression of the Arc protein in a host cell. It will be appreciated by those skilled in the art that the design of the vector, including the selection of regulatory sequences can depend on such factors as the choice of the host cell to be transformed, the level of expression of protein desired, etc. Preferred regulatory sequences for mammalian host cell expression include viral elements that direct high levels of protein expression in mammalian cells, such as promoters and/or enhancers derived from retroviral LTRs, cytomegalovirus (CMV) (such as the CMV promoter/enhancer), Simian Virus 40 (SV40) (such as the SV40 promoter/enhancer), adenovirus, (e.g., the adenovirus major late promoter (AdMLP)), polyoma and strong mammalian promoters such as native immunoglobulin and actin promoters. For further description of viral regulatory elements, and sequences thereof, see e.g., U.S. Pat. Nos. 5,168,062, 4,510,245 and 4,968,615. Methods of expressing polypeptides in bacterial cells or fungal cells, e.g., yeast cells, are also well known in the art.

In some apsects, the disclosed vectors further comprise a promoter operably linked to the nucleic acid sequence capable of encoding the Arc protein. In some apsects, the promoter can be an inducible promoter. In some apsects, the promoter can be a cell-specific promoter. The nucleic acid sequence capable of encoding the Arc protein can be functionally linked to a promoter. By “functionally linked” is meant such that the promoter can promote expression of the nucleic acid sequence, thus having appropriate orientation of the promoter relative to the nucleic acid sequence.

### D. Cells

Disclosed are cells comprising any of the disclosed vectors. Disclosed are cells comprising any of the disclosed Arc proteins. Disclosed are cells comprising any of the disclosed Arc capsids.

In some aspects, the disclosed cells can be mammalian cells.

In some instances, cells can be cultured using culturing techniques well known in the art. Any known cell lines can be used. In some instances, cells can be derived from any host. For example, cells can be derived from, but are not limited to, a human, rat, mouse, dog, cat, horse, bacteria, or fungi host.

### E. Pharmaceutical Compositions

Disclosed are compositions comprising an Arc capsid and a pharmaceutically acceptable carrier. The Arc capsid can be any of the disclosed Arc capsids.

In some aspects, the disclosed Arc capsids can be formulated and/or administered in or with a pharmaceutically acceptable carrier. As used herein, the term “pharmaceutically acceptable carrier” refers to sterile aqueous or nonaqueous solutions, dispersions, suspensions or emulsions, as well as sterile powders for reconstitution into sterile injectable solutions or dispersions just prior to use. Examples of suitable aqueous and nonaqueous carriers, diluents, solvents or vehicles include water, ethanol, polyols (such as glycerol, propylene glycol, polyethylene glycol and the like), carboxymethylcellulose and suitable mixtures thereof, vegetable oils (such as olive oil) and injectable organic esters such as ethyl oleate. Proper fluidity can be maintained, for example, by the use of coating materials such as lecithin, by the maintenance of the required particle size in the case of dispersions and by the use of surfactants. These compositions can also contain adjuvants such as preservatives, wetting agents, emulsifying agents and dispersing agents. Prevention of the action of microorganisms can be ensured by the inclusion of various antibacterial and antifungal agents such as paraben, chlorobutanol, phenol, sorbic acid and the like. It can also be desirable to include isotonic agents such as sugars, sodium chloride and the like. Prolonged absorption of the injectable pharmaceutical form can be brought about by the inclusion of agents, such as aluminum monostearate and gelatin, which delay absorption. Injectable depot forms are made by forming microencapsule matrices of the drug in biodegradable polymers such as polylactide-polyglycolide, poly(orthoesters) and poly(anhydrides). Depending upon the ratio of drug to polymer and the nature of the particular polymer employed, the rate of drug release can be controlled. Depot injectable formulations are also prepared by entrapping the drug in liposomes or microemulsions that are compatible with body tissues. The injectable formulations can be sterilized, for example, by filtration through a bacterial-retaining filter or by incorporating sterilizing agents in the form of sterile solid compositions which can be dissolved or dispersed in sterile water or other sterile injectable media just prior to use. Suitable inert carriers can include sugars such as lactose. Desirably, at least 95% by weight of the particles of the active ingredient have an effective particle size in the range of 0.01 to 10 micrometers.

Thus, the compositions disclosed herein can comprise lipids such as liposomes, such as cationic liposomes (e.g., DOTMA, DOPE, DC-cholesterol) or anionic liposomes. Liposomes can further comprise proteins to facilitate targeting a particular cell, if desired. Administration of a composition comprising a peptide and a cationic liposome can be administered to the blood, to a target organ, or inhaled into the respiratory tract to target cells of the respiratory tract. For example, a composition comprising a peptide or nucleic acid sequence described herein and a cationic liposome can be administered to a subject's lung cells. Regarding liposomes, see, e.g., Brigham et al. Am. J. Resp. Cell. Mol. Biol. 1:95 100 (1989); Felgner et al. Proc. Natl. Acad. Sci USA 84:7413 7417 (1987); U.S. Pat. No. 4,897,355. Furthermore, the compound can be administered as a component of a microcapsule that can be targeted to specific cell types, such as macrophages, or where the diffusion of the compound or delivery of the compound from the microcapsule is designed for a specific rate or dosage.

In one aspect, disclosed are pharmaceutical compositions comprising any of the disclosed Arc capsids or proteins described herein, or a pharmaceutically acceptable salt or solvate thereof, and a pharmaceutically acceptable carrier, buffer, or diluent. In various aspects, the Arc capsid or protein of the pharmaceutical composition is encapsulated in a delivery vehicle. In a further aspect, the delivery vehicle is a liposome, a microcapsule, or a nanoparticle. In a still further aspect, the delivery vehicle is PEG-ylated.

In the methods described herein, delivery of the compositions to cells can be via a variety of mechanisms. As defined above, disclosed herein are compositions comprising any one or more of the Arc capsids or proteins described herein and can also include a carrier such as a pharmaceutically acceptable carrier. For example, disclosed are pharmaceutical compositions, comprising the Arc capsids and proteins disclosed herein, and a pharmaceutically acceptable carrier. In one aspect, disclosed are pharmaceutical compositions comprising the disclosed Arc capsids and proteins. That is, a pharmaceutical composition can be provided comprising a therapeutically effective amount of at least one disclosed Arc capsid or at least one product of a disclosed method and a pharmaceutically acceptable carrier.

In certain aspects, the disclosed pharmaceutical compositions comprise the disclosed Arc capsids or proteins (including pharmaceutically acceptable salt(s) thereof) as an active ingredient, a pharmaceutically acceptable carrier, and, optionally, other therapeutic ingredients or adjuvants. The instant compositions include those suitable for oral, rectal, topical, and parenteral (including subcutaneous, intramuscular, and intravenous) administration, although the most suitable route in any given case will depend on the particular host, and nature and severity of the conditions for which the active ingredient is being administered. The pharmaceutical compositions can be conveniently presented in unit dosage form and prepared by any of the methods well known in the art of pharmacy.

In practice, the Arc capsids and proteins described herein, or pharmaceutically acceptable salts thereof, of this invention can be combined as the active ingredient in intimate admixture with a pharmaceutical carrier according to conventional pharmaceutical compounding techniques. The carrier can take a wide variety of forms depending on the form of preparation desired for administration, e.g., oral or parenteral (including intravenous). Thus, the pharmaceutical compositions of the present invention can be presented as discrete units suitable for oral administration such as capsules, cachets or tablets each containing a predetermined amount of the active ingredient. Further, the compositions can be presented as a powder, as granules, as a solution, as a suspension in an aqueous liquid, as a non-aqueous liquid, as an oil-in-water emulsion or as a water-in-oil liquid emulsion. In addition to the common dosage forms set out above, the compounds of the invention, and/or pharmaceutically acceptable salt(s) thereof, can also be administered by controlled release means and/or delivery devices. The compositions can be prepared by any of the methods of pharmacy. In general, such methods include a step of bringing into association the active ingredient with the carrier that constitutes one or more necessary ingredients. In general, the compositions are prepared by uniformly and intimately admixing the active ingredient with liquid carriers or finely divided solid carriers or both. The product can then be conveniently shaped into the desired presentation.

By “pharmaceutically acceptable” is meant a material or carrier that would be selected to minimize any degradation of the active ingredient and to minimize any adverse side effects in the subject, as would be well known to one of skill in the art. The Arc capsids or proteins described herein, or pharmaceutically acceptable salts thereof, can also be included in pharmaceutical compositions in combination with one or more other therapeutically active compounds.

The pharmaceutical carrier employed can be, for example, a solid, liquid, or gas. Examples of solid carriers include lactose, terra alba, sucrose, talc, gelatin, agar, pectin, acacia, magnesium stearate, and stearic acid. Examples of liquid carriers are sugar syrup, peanut oil, olive oil, and water. Examples of gaseous carriers include carbon dioxide and nitrogen. Other examples of carriers include dimyristoylphosphatidyl (DMPC), phosphate buffered saline or a multivesicular liposome. For example, PG:PC:Cholesterol:peptide or PC:peptide can be used as carriers in this invention. Other suitable pharmaceutically acceptable carriers and their formulations are described in Remington: The Science and Practice of Pharmacy (19th ed.) ed. A. R. Gennaro, Mack Publishing Company, Easton, Pa. 1995. Typically, an appropriate amount of pharmaceutically-acceptable salt is used in the formulation to render the formulation isotonic. Other examples of the pharmaceutically-acceptable carrier include, but are not limited to, saline, Ringer's solution and dextrose solution. The pH of the solution can be from about 5 to about 8, or from about 7 to about 7.5. Further carriers include sustained release preparations such as semi-permeable matrices of solid hydrophobic polymers containing the composition, which matrices are in the form of shaped articles, e.g., films, stents (which are implanted in vessels during an angioplasty procedure), liposomes or microparticles. It will be apparent to those persons skilled in the art that certain carriers may be more preferable depending upon, for instance, the route of administration and concentration of composition being administered. These most typically would be standard carriers for administration of drugs to humans, including solutions such as sterile water, saline, and buffered solutions at physiological pH.

In order to enhance the solubility and/or the stability of the disclosed Arc capsids or proteins in pharmaceutical compositions, it can be advantageous to employ α-, β- or γ-cyclodextrins or their derivatives, in particular hydroxyalkyl substituted cyclodextrins, e.g. 2-hydroxypropyl-β-cyclodextrin or sulfobutyl-β-cyclodextrin. Also co-solvents such as alcohols may improve the solubility and/or the stability of the compounds according to the invention in pharmaceutical compositions.

Pharmaceutical compositions can also include carriers, thickeners, diluents, buffers, preservatives and the like, as long as the intended activity of the polypeptide, peptide, nucleic acid, vector of the invention is not compromised. Pharmaceutical compositions may also include one or more active ingredients (in addition to the composition of the invention) such as antimicrobial agents, anti-inflammatory agents, anesthetics, and the like. The pharmaceutical composition may be administered in a number of ways depending on whether local or systemic treatment is desired, and on the area to be treated.

Because of the ease in administration, oral administration can be used, and tablets and capsules represent the most advantageous oral dosage unit forms in which case solid pharmaceutical carriers are obviously employed. In preparing the compositions for oral dosage form, any convenient pharmaceutical media can be employed. For example, water, glycols, oils, alcohols, flavoring agents, preservatives, coloring agents and the like can be used to form oral liquid preparations such as suspensions, elixirs and solutions; while carriers such as starches, sugars, microcrystalline cellulose, diluents, granulating agents, lubricants, binders, disintegrating agents, and the like can be used to form oral solid preparations such as powders, capsules and tablets. Because of their ease of administration, tablets and capsules are the preferred oral dosage units whereby solid pharmaceutical carriers are employed. Optionally, tablets can be coated by standard aqueous or nonaqueous techniques.

Compositions for oral administration include powders or granules, suspensions or solutions in water or non-aqueous media, capsules, sachets, or tablets. Thickeners, flavorings, diluents, emulsifiers, dispersing aids, or binders may be desirable. Some of the compositions may potentially be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mon-, di-, trialkyl and aryl amines and substituted ethanolamines.

A tablet containing the compositions of the present invention can be prepared by compression or molding, optionally with one or more accessory ingredients or adjuvants. Compressed tablets can be prepared by compressing, in a suitable machine, the active ingredient in a free-flowing form such as powder or granules, optionally mixed with a binder, lubricant, inert diluent, surface active or dispersing agent. Molded tablets can be made by molding in a suitable machine, a mixture of the powdered compound moistened with an inert liquid diluent.

The pharmaceutical compositions of the present invention comprise a protein such as an Arc protein or capsid (or pharmaceutically acceptable salts thereof) as an active ingredient, a pharmaceutically acceptable carrier, and optionally one or more additional therapeutic agents or adjuvants. The instant compositions include compositions suitable for oral, rectal, topical, and parenteral (including subcutaneous, intramuscular, and intravenous) administration, although the most suitable route in any given case will depend on the particular host, and nature and severity of the conditions for which the active ingredient is being administered. The pharmaceutical compositions can be conveniently presented in unit dosage form and prepared by any of the methods well known in the art of pharmacy.

Pharmaceutical compositions of the present invention suitable for parenteral administration can be prepared as solutions or suspensions of the active compounds in water. A suitable surfactant can be included such as, for example, hydroxypropylcellulose. Dispersions can also be prepared in glycerol, liquid polyethylene glycols, and mixtures thereof in oils. Further, a preservative can be included to prevent the detrimental growth of microorganisms.

Pharmaceutical compositions of the present invention suitable for injectable use include sterile aqueous solutions or dispersions. Furthermore, the compositions can be in the form of sterile powders for the extemporaneous preparation of such sterile injectable solutions or dispersions. Typically, the final injectable form should be sterile and should be effectively fluid for easy syringability. The pharmaceutical compositions should be stable under the conditions of manufacture and storage; thus, preferably should be preserved against the contaminating action of microorganisms such as bacteria and fungi. The carrier can be a solvent or dispersion medium containing, for example, water, ethanol, polyol (e.g., glycerol, propylene glycol and liquid polyethylene glycol), vegetable oils, and suitable mixtures thereof.

Injectable solutions, for example, can be prepared in which the carrier comprises saline solution, glucose solution or a mixture of saline and glucose solution. Injectable suspensions may also be prepared in which case appropriate liquid carriers, suspending agents and the like may be employed. Also included are solid form preparations that are intended to be converted, shortly before use, to liquid form preparations.

Preparations of parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

Pharmaceutical compositions of the present invention can be in a form suitable for topical use such as, for example, an aerosol, cream, ointment, lotion, dusting powder, mouth washes, gargles, and the like. Further, the compositions can be in a form suitable for use in transdermal devices. These formulations can be prepared, utilizing a compound of the invention, or pharmaceutically acceptable salts thereof, via conventional processing methods. As an example, a cream or ointment is prepared by mixing hydrophilic material and water, together with about 5 wt % to about 10 wt % of the compound, to produce a cream or ointment having a desired consistency.

In the compositions suitable for percutaneous administration, the carrier optionally comprises a penetration enhancing agent and/or a suitable wetting agent, optionally combined with suitable additives of any nature in minor proportions, which additives do not introduce a significant deleterious effect on the skin. Said additives may facilitate the administration to the skin and/or may be helpful for preparing the desired compositions. These compositions may be administered in various ways, e.g., as a transdermal patch, as a spot on, as an ointment.

Pharmaceutical compositions of this invention can be in a form suitable for rectal administration wherein the carrier is a solid. It is preferable that the mixture forms unit dose suppositories. Suitable carriers include cocoa butter and other materials commonly used in the art. The suppositories can be conveniently formed by first admixing the composition with the softened or melted carrier(s) followed by chilling and shaping in molds.

Formulations for optical administration may include ointments, lotions, creams, gels, drops, suppositories, sprays, liquids and powders. Conventional pharmaceutical carriers, aqueous, powder or oily bases, thickeners and the like may be desirable.

In addition to the aforementioned carrier ingredients, the pharmaceutical formulations described above can include, as appropriate, one or more additional carrier ingredients such as diluents, buffers, flavoring agents, binders, surface-active agents, thickeners, lubricants, preservatives (including anti-oxidants) and the like. Furthermore, other adjuvants can be included to render the formulation isotonic with the blood of the intended recipient. Compositions containing a disclosed peptide, and/or pharmaceutically acceptable salts thereof, can also be prepared in powder or liquid concentrate form.

The exact dosage and frequency of administration depends on the particular disclosed Arc capsid or protein, a product of a disclosed method of making, a pharmaceutically acceptable salt, solvate, or polymorph thereof, a hydrate thereof, a solvate thereof, a polymorph thereof, or a stereochemically isomeric form thereof; the particular condition being treated and the severity of the condition being treated; various factors specific to the medical history of the subject to whom the dosage is administered such as the age; weight, sex, extent of disorder and general physical condition of the particular subject, as well as other medication the individual may be taking; as is well known to those skilled in the art. Furthermore, it is evident that said effective daily amount may be lowered or increased depending on the response of the treated subject and/or depending on the evaluation of the physician prescribing the compositions.

Depending on the mode of administration, the pharmaceutical composition will comprise from 0.05 to 99% by weight, preferably from 0.1 to 70% by weight, more preferably from 0.1 to 50% by weight of the active ingredient, and, from 1 to 99.95% by weight, preferably from 30 to 99.9% by weight, more preferably from 50 to 99.9% by weight of a pharmaceutically acceptable carrier, all percentages being based on the total weight of the composition.

### F. Methods of Delivering mRNA

Disclosed are methods of delivering mRNA to a cell comprising administering an Arc capsid to a cell, wherein the Arc capsid comprises an mRNA of interest. The term “mRNA sequence if interest” or “mRNA of interest” can mean an mRNA nucleic acid sequence (e.g., a therapeutic gene), that is partly or entirely heterologous, i.e., foreign, to a cell into which it is introduced. The term “mRNA sequence if interest” or “mRNA of interest” can also mean an mRNA nucleic acid sequence, that is partly or entirely homologous to an endogenous gene of the cell into which it is introduced, but which is designed to be introduced to a cell. The term “mRNA sequence if interest” or “mRNA of interest” can also mean an mRNA nucleic acid sequence, that is partly or entirely complementary to an endogenous gene of the cell into which it is introduced. For example, the mRNA sequence of interest can be micro RNA, shRNA, or siRNA. An “mRNA sequence if interest” or “mRNA of interest” can also include one or more transcriptional regulatory sequences and any other nucleic acid, such as introns, that may be necessary for optimal expression of a selected nucleic acid.

Any of the disclosed Arc capsids can be used in these methods described herein.

In some aspects, the Arc capsid can be heterologous to the cell. In some aspects, an Arc capsid that is heterologous to the cell is any Arc capsid that was not derived from the cell to which it is being delivered.

Disclosed are methods of delivering mRNA to a cell comprising administering any one or more of the disclosed vectors to a cell; and administering an mRNA of interest to the cell; wherein a nucleic acid sequence within the vector encodes an Arc protein that is then translated within the cell and Arc capsids are formed, wherein the Arc capsids encapsulate the mRNA of interest.

In some aspects of the disclosed methods of delivery, the cell can be a neuron. In some aspects, the cell can be a mammalian cell, such as, but not limited to, a human cell. In some aspects, the cell can be, but is not limited to, a nerve cell, a muscle cell, a bone cell, a gland cell, a blood cell, or a reproductive cell. For example, the cell can be a T cell, a B cell, a macrophage, an epithelial cell, a chondrocyte or a stem cell.

In some aspects, the mRNA of interest is a therapeutic. For example, the therapeutic can be, but is not limited to, an immunomodulatory agent such as cytokines encoded by the mRNA, siRNA, or an inhibitor encoded by the mRNA. Thus, in some aspects, the Arc capsid is used to deliver a therapeutic to a cell and the therapeutic is able to treat any condition in the cell. For example, a condition in a cell can be anything caused by a disease or disorder in which a subject has been diagnosed with and the cell is from the said subject. In some aspects, the mRNA of interest is not Arc mRNA.

In some aspects, the vector comprises the mRNA of interest. For example, in some aspects the vector comprising the nucleic acid sequence capable of encoding an Arc protein can further comprise the mRNA of interest. In some aspects, the mRNA of interest can be administered in a second vector that is separate from the vector comprising the nucleic acid sequence capable of encoding an Arc protein.

Disclosed are methods of delivering mRNA to a subject comprising administering one or more of any one of the disclosed cells to a subject in need thereof. In some aspects, the cells can be heterologous. In some aspects, the cell can be autologous.

Disclosed herein are methods of delivering an mRNA of interest to a subject comprising exposing cells obtained from a subject to any one of the disclosed Arc capsids comprising an mRNA sequence of interest, wherein the cells exposed to the Arc capsid take up the Arc capsid forming cells comprising the Arc capsid comprising an mRNA of interest; and administering the cells comprising the Arc capsid comprising an mRNA of interest to the subject from which the cells were obtained. In some aspects, the Arc capsids comprise a heterologous mRNA sequence. A heterologous mRNA sequence can be any mRNA sequence that is not derived from the same cell as the Arc capsid. In some aspects, the heterologous mRNA sequence is a non-Arc mRNA sequence.

Disclosed herein are methods of delivering an mRNA of interest to a subject comprising exposing cells obtained from a subject to any one of the disclosed Arc capsids comprising an mRNA sequence of interest, wherein the cells exposed to the Arc capsid take up the Arc capsid forming cells comprising the Arc capsid comprising an mRNA of interest; and administering the cells comprising the Arc capsid comprising an mRNA of interest to a subject other than the subject from which the cells were obtained.

### G. Methods of Forming Arc Capsids

Disclosed are methods of forming Arc capsids comprising administering any of the disclosed vectors to a solution comprising cells, wherein the nucleic acid sequence encodes an Arc protein within the cells and Arc capsids are formed.

Disclosed are methods of forming Arc capsids comprising administering any of the disclosed vectors to a solution comprising cells, wherein the nucleic acid sequence encodes an Arc protein within the cells and Arc capsids are formed, further comprising administering a mRNA of interest, wherein the mRNA is packaged in the Arc capsids during Arc formation.

In some aspects, the disclosed methods of forming Arc capsids further comprises increasing the salt concentration of the solution to a range of 100 mM to 300 mM. In some aspects, the salt can be, but is not limited to, NaCl or NaPO4.

In some aspects, the Arc capsids that are formed are released from the cell via extracellular vesicles. In some aspects, the cells are recombinant cells comprising a cell membrane which is involved in the forming of the extracellular vesicle, wherein the extracellular vesicle provides cell specificity for targeting of the Arc capsid. In some aspects, the Arc capsids can be formed in the presence of an exogenous nucleic acid which is capable of controlling Arc capsid assembly.

In some aspects, the Arc capsid can be produced or delivered via exosomes or extracellular vesicles made in cells. For example, exosomes or extracellular vesicles can be used as potential vectors for Arc capsid production and dissemination.

### H. Method of Blocking Arc Binding

A method of blocking Arc capsid binding to a lipid comprising administering a blocking agent, wherein the blocking agent interrupts the binding of an Arc capsid to a lipid. In some aspects the blocking agent can be any molecule that binds the Arc capsid and blocks the lipid binding site or binds the lipid and blocks the Arc capsid binding site. In some aspects, the blocking agent can be an Arc protein or fragment thereof.

### I. Kits

The materials described above as well as other materials can be packaged together in any suitable combination as a kit useful for performing, or aiding in the performance of, the disclosed method. It is useful if the kit components in a given kit are designed and adapted for use together in the disclosed method. For example disclosed are kits for producing Arc capsids, the kit comprising any of the disclosed Arc proteins, Arc nucleic acids, vectors or cells.

### Examples

Brains have evolved to process and store information from the outside world through synaptic connections between interconnected networks of neurons. Despite the fundamental importance of information storage in the brain, a detailed molecular and cellular understanding of the processes involved and their evolutionary origins is still lacking. Studies over the last decade have shown that eukaryotic genomes are littered with DNA of viral or transposon origin, which comprise about half of most mammalian genomes (Smit, 1999). There is growing appreciation that the sequences encoded by these elements can provide raw material for the emergence of new functions and regulatory elements (Chuong et al., 2017; Levin and Moran, 2011). In vertebrates, these include dozens of protein-coding genes derived from sequences previously encoded by transposons (Feschotte and Pritham, 2007; Naville et al., 2016) or retroviruses (Kaneko-Ishino and Ishino, 2012). Interestingly, many of these transposon-derived genes are expressed in the brain, but their molecular functions remain to be elucidated.

The neuronal gene Arc contains structural elements found within viral Group-specific antigen (Gag) polyproteins that may have originated from the Ty3/gypsy retrotransposon family (Campillos et al., 2006; Day and Shepherd, 2015; Zhang et al., 2015), although the role these Gag elements play in Arc function has not been explored. Arc is a master regulator of synaptic plasticity in mammals and is required for protein synthesis-dependent forms of long-term potentiation (LTP) and depression (LTD) (Bramham et al., 2010; Shepherd and Bear, 2011). Arc can regulate synaptic plasticity through the trafficking of AMPA-type glutamate receptors (AMPARs) via the endocytic machinery (Chowdhury et al., 2006). This endocytic pathway maintains levels of surface AMPARs in response to chronic changes in neuronal activity through synaptic scaling, thus contributing to homeostasis of neuronal strength (Shepherd et al., 2006). Arc's expression in the brain is highly dynamic; its transcription is tightly coupled to encoding of information in neuronal circuits in vivo (Guzowski et al., 1999). Arc mRNA is transported to dendrites and becomes enriched at sites of local synaptic activity where it is locally translated into protein (Steward et al., 1998; Waung et al., 2008). Intriguingly, aspects of Arc mRNA regulation resemble some viral RNAs, as Arc contains an internal ribosomal entry site (IRES) that allows cap-independent translation (Balvay et al., 2007; Pinkstaff et al., 2001). In vivo, Arc is required to transduce experience into long-lasting changes in visual cortex plasticity (McCurry et al., 2010) and for long-term memory (Guzowski et al., 2000; Plath et al., 2006). In addition, Arc has been implicated in various neurological disorders that include Alzheimer's disease (Wu et al., 2011), monogenic forms of intellectual disability such as Angelman (Greer et al., 2010; Pastuzyn and Shepherd, 2017) and Fragile-X Syndromes (Park et al., 2008), and schizophrenia (Fromer et al., 2014; Manago et al., 2016; Purcell et al., 2014). Thus, precise regulation of Arc expression and activity in the nervous system seems essential for normal cognition.

Despite its importance, little is known about Arc protein biochemistry and molecular function. One role for Arc is mediating intercellular communication via extracellular vesicles (EVs). Synaptic communication is supplemented or modulated by many other communication pathways that include glia-neuron interactions, and emerging evidence suggests that EVs mediate intercellular signaling in the nervous system (Budnik et al., 2016; Zappulli et al., 2016). EVs can be broadly defined into two groups, microvesicles and exosomes, which are defined both by the size of the EV and the subcellular origin. Microvesicles pinch off from the plasma membrane directly and are usually 100-300 nm in diameter, whereas exosomes are derived from intraluminal vesicles that originate from multivesicular bodies (MVBs) and are usually <100 nm in size. EVs can transport cargo that do not readily cross the plasma membrane, such as membrane proteins and various forms of RNA. The observation that EVs can function in the intercellular transport of these molecules within the nervous system opens an entirely new perspective on intercellular communication in the brain.

Arc protein self-assembles into oligomers that resemble virus capsids and exhibits several other biochemical properties seen in retroviral Gag proteins such as lipid and RNA binding. Moreover, Arc is released from neurons in EVs and is able to transfer its own mRNA into neurons. The Drosophila Arc homologue, dArc1, also forms capsids and mediates intercellular transfer of its own mRNA at the fly neuromuscular junction, despite originating from a distinct retrotransposon lineage. These data indicate that co-option of retroviral-like Gag elements may have provided an evolutionary pathway for novel mechanisms that mediate intercellular signaling and have been intricately involved in the evolution of synaptic plasticity and animal cognition.

1. Results

i. Fly and Tetrapod Arc Genes Originated Independently from Distinct Lineages of Ty3/Gypsy Retrotransposons

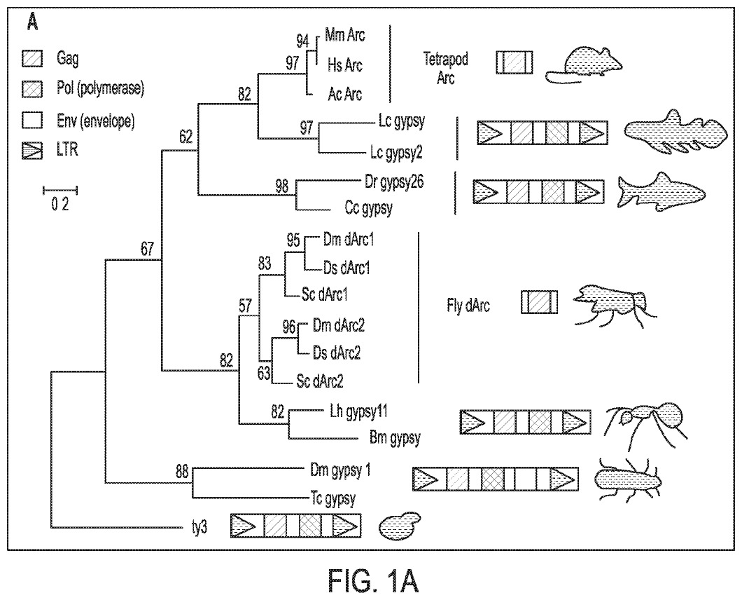

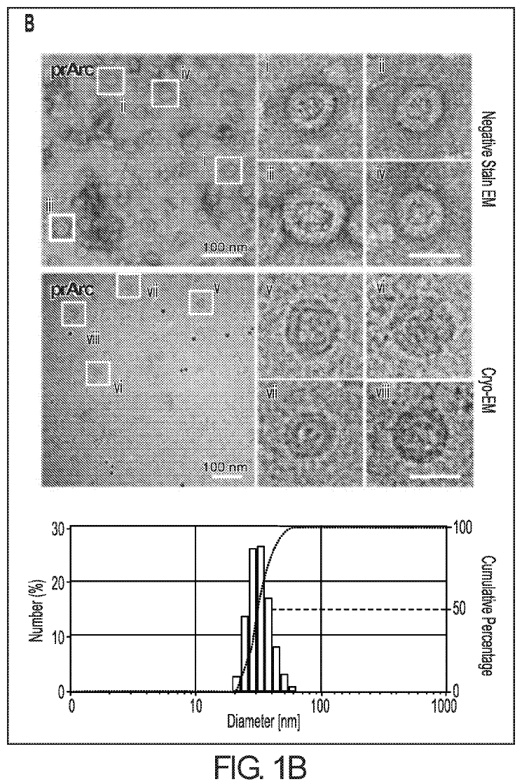

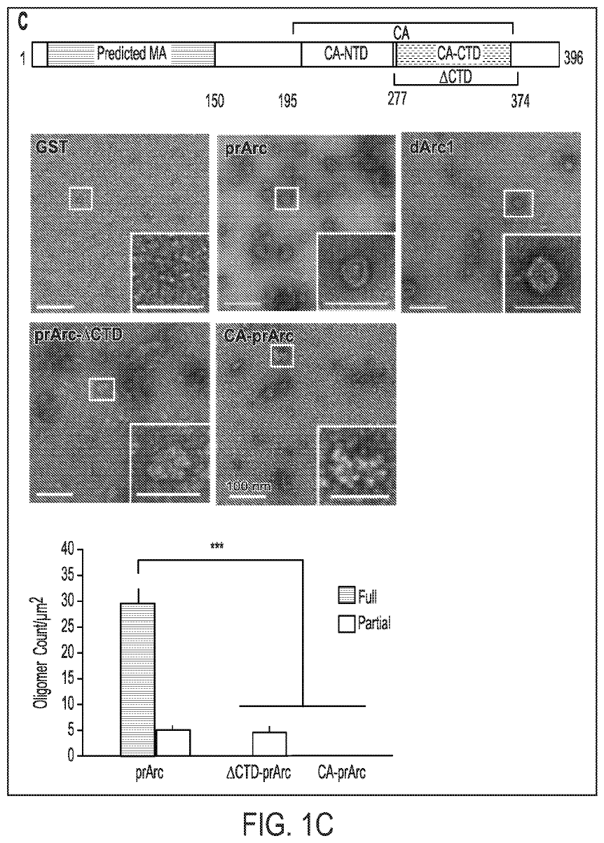

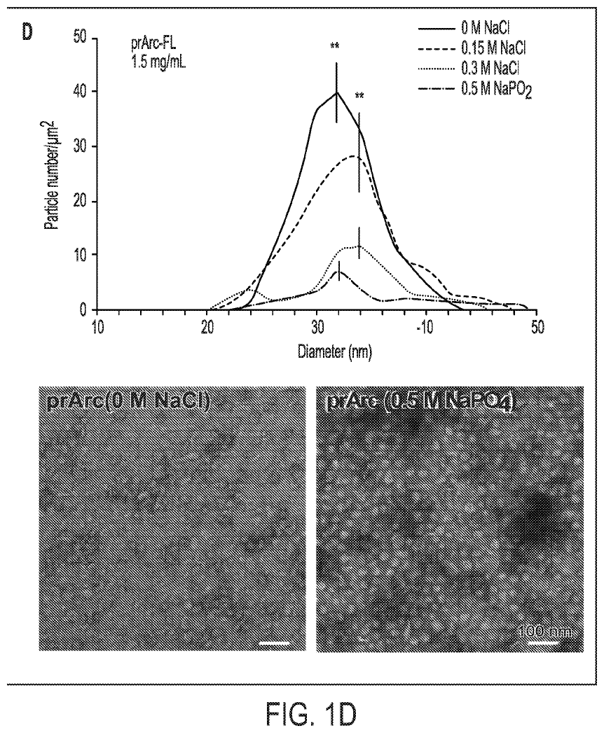

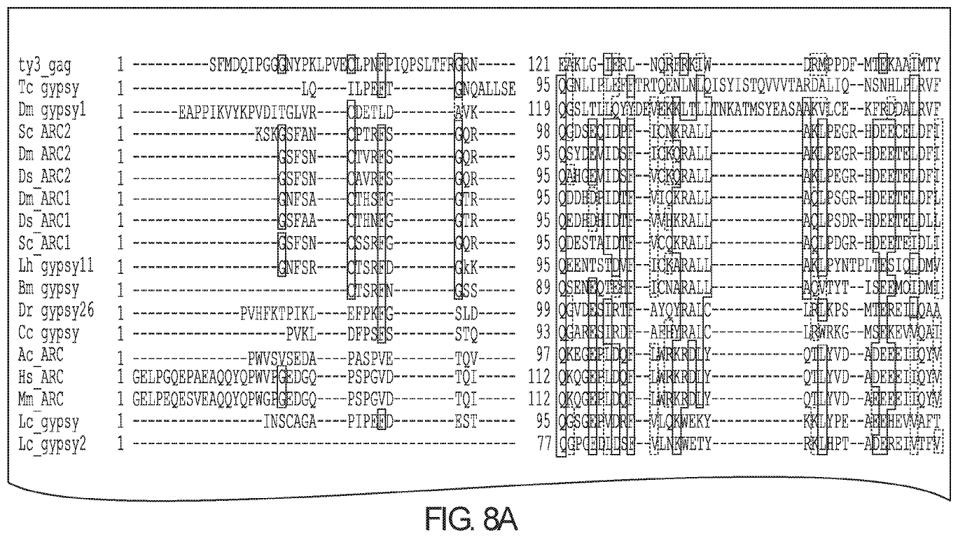

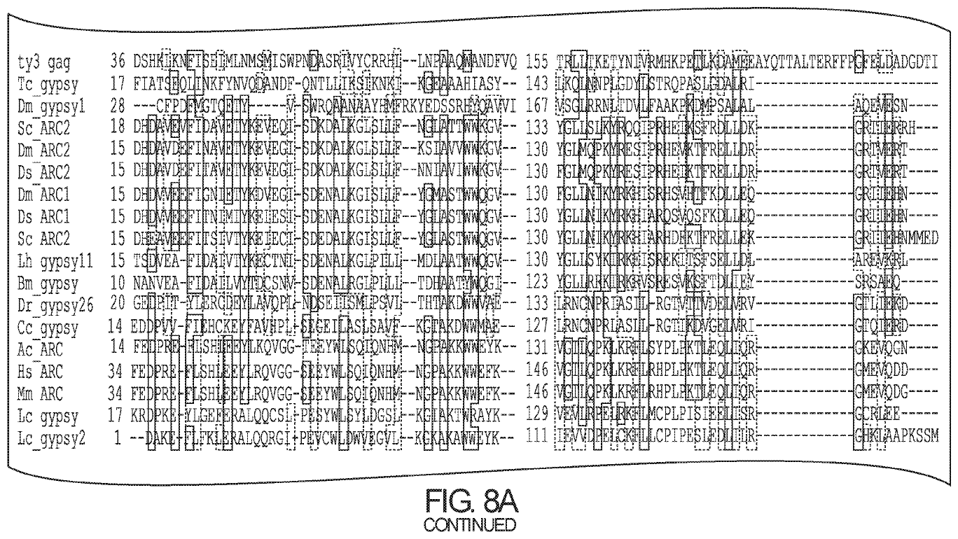

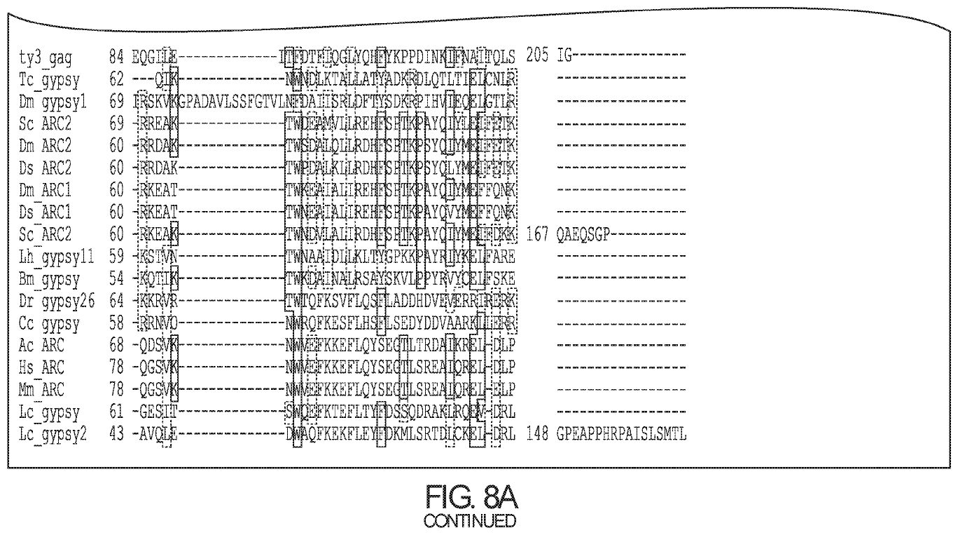

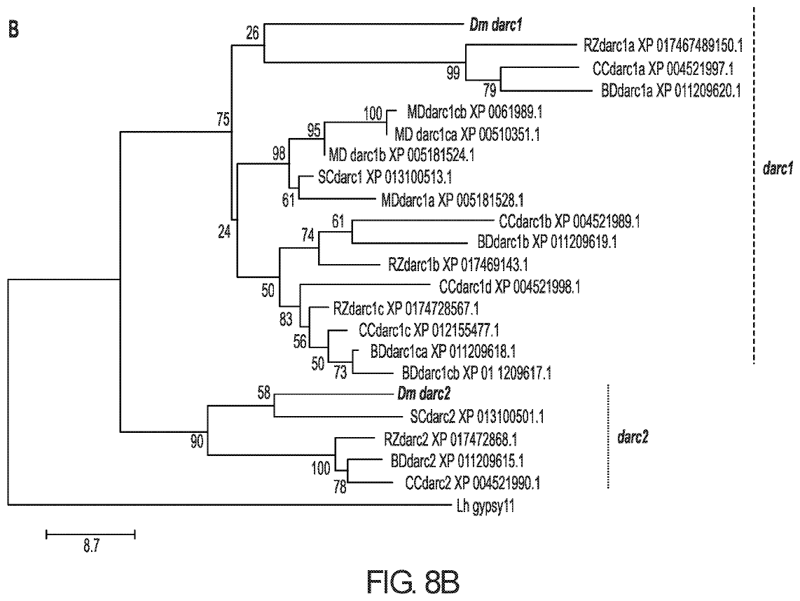

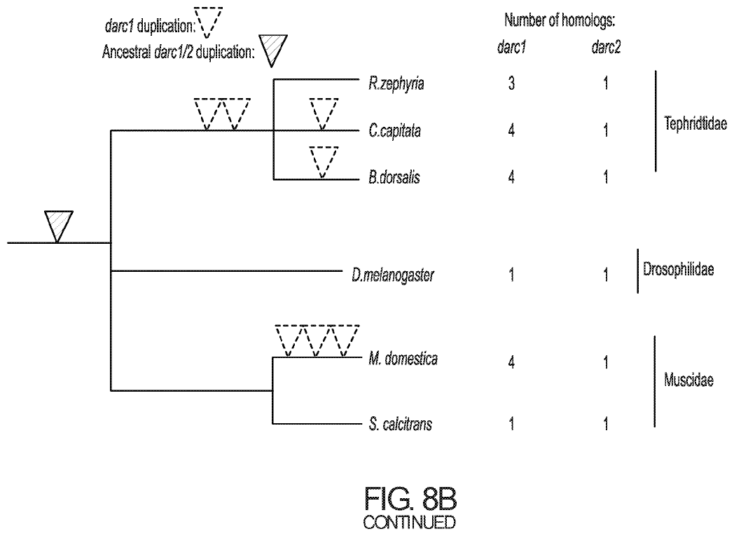

To shed light onto Arc's evolutionary origins, phylogenomic analyses were performed (FIGS. 1A and 8A). Highly conserved, unique orthologs of the murine Arc genes were identified throughout the tetrapods (mammals, birds, reptiles, amphibians), but were conspicuously absent from all fish lineages and other deuterostomes examined (94 species). The closest relatives of Arc in the coelacanth, zebrafish, and carp genomes were encoded by prototypical Ty3/gypsy retrotransposons, with indications of recent transposition activity. Similarly, orthologs and paralogs of Drosophila Arc (darc1, darc2) were identified in all schizophoran (true) flies represented in the database but were not detected in any other dipteran (e.g., mosquitoes) or protostome species (286 species; FIG. 8B). The closest retrotransposon relatives of the fly Arc genes were found in the genomes of the silkworm and Argentine ant. Interestingly, while Arc appears to be a single-copy gene in all tetrapods examined, the gene has experienced multiple rounds of duplication during schizophoran evolution (FIG. 8B). Phylogenetically, tetrapod Arc genes cluster with Ty3/gypsy retrotransposons from fish, whereas the fly Arc homologs group with a separate lineage of Ty3/gypsy retrotransposons from insects (FIG. 1A). These results indicate that the tetrapod and fly Arc genes originated independently from distinct lineages of Ty3/gypsy retrotransposons, as conjectured previously (Abrusán et al., 2013), but still share significant homology in the retroviral Gag domain.

ii. Arc Proteins Self-Assemble into Virus-Like Capsids

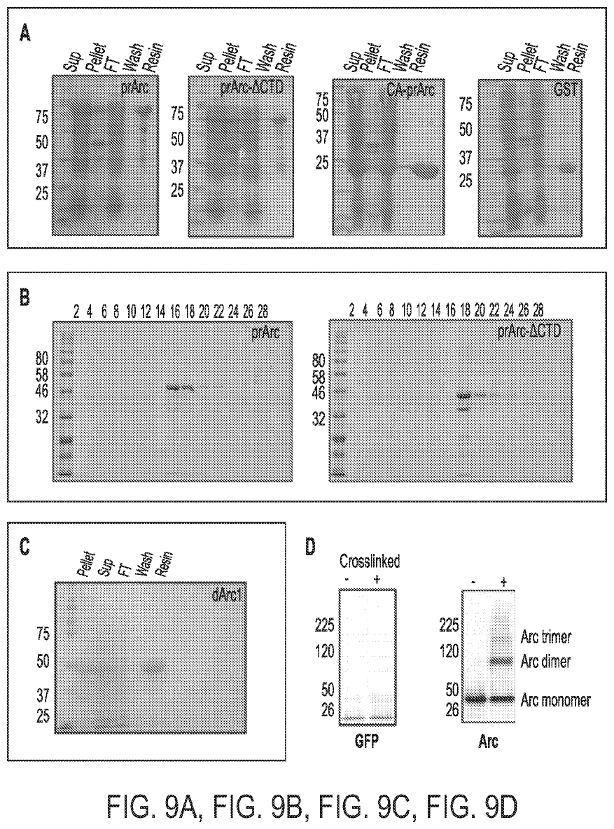

Ty3 retrotransposons can form oligomeric particles that resemble retroviral capsids (Hansen et al., 1992), and Arc also has a propensity to oligomerize (Myrum et al., 2015). Retroviral capsid formation is essential for infectivity and is primarily mediated by the Gag polyprotein, which in HIV contains four main functional domains: matrix/MA, capsid/CA, nucleocapsid/NC, and p6 (Freed, 2015). Arc has both primary sequence (Campillos et al., 2006) and structural similarity to CA of HIV and Foamy Virus Gag polyproteins (Taylor et al., 2017; Zhang et al., 2015), suggesting that Arc may share functional similarities to Gag proteins. To characterize the biochemical properties of Arc protein, rat Arc was expressed in bacteria as a glutathione S-transferase (GST) fusion protein. The expressed protein was purified by affinity and size exclusion chromatography, and the GST tag was removed by proteolysis (FIGS. 9A and 9B). Purified preparations of rat Arc (prArc) were analyzed using negative-stain electron microscopy (EM) and cryoelectron microscopy (cryo-EM). These experiments revealed that prArc spontaneously forms oligomeric structures that resemble virus-like capsids (FIG. 1B). prArc capsids exhibited a double-shell structure with a mean diameter of 32±0.2 nm. Similarly, bacterially expressed and purified dArc1 (FIG. 9C), the Drosophila Arc homolog, also self-assembled into capsid-like structures (FIG. 1C). Purified Arc protein that was expressed in an insect cell expression system also assembled into similar virus-like capsids (data not shown), indicating that oligomerization was not an artifact of bacterial expression. Immature retroviral capsids are formed by the uncleaved Gag polyprotein, and the major stabilizing interactions are made by the C-terminal domain (CTD) of the CA region (Maffei et al., 2016). To test whether the putative Arc CA CTD is also required for self-assembly, a rat Arc mutant protein that lacked this domain (prArc-ΔCTD, missing amino acids [aa] 277-374, FIGS. 1C, 9A, and 9B) was expressed and purified (Zhang et al., 2015). EM analyses revealed that prArc-ΔCTD was unable to form double-shelled capsids, although intermediate irregular structures were occasionally observed (FIG. 1C).

To test whether the Arc CA domain was sufficient for capsid assembly a mutant Arc protein was created that contained aa195-364 (CA-prArc; FIGS. 1C and 9A). CA-prArc was not sufficient to form capsid-like structures. Arc capsids exhibit other properties similar to HIV capsids, including sensitivity to salt and phosphate levels (Purdy et al., 2008); increasing concentrations of NaCl from 0 to 300 mM resulted in stable prArc capsids and high NaPO4 further stabilized capsid formation (FIG. 1D). To test whether Arc forms oligomers in cells, Arc was expressed in HEK293 cells, which lack endogenous Arc, and performed chemical crosslinking to test for the presence of oligomeric species. Arc proteins crosslinked in situ formed higher molecular weight species with the SDS-PAGE mobility expected for dimer and trimer subunits (FIG. 9D), which is reminiscent of HIV Gag subunits using a similar crosslinking assay (Campbell and Rein, 1999). In contrast, transfected GFP did not form higher molecular weight crosslinks under the same conditions.

iii. Arc Binds and Encapsulates RNA

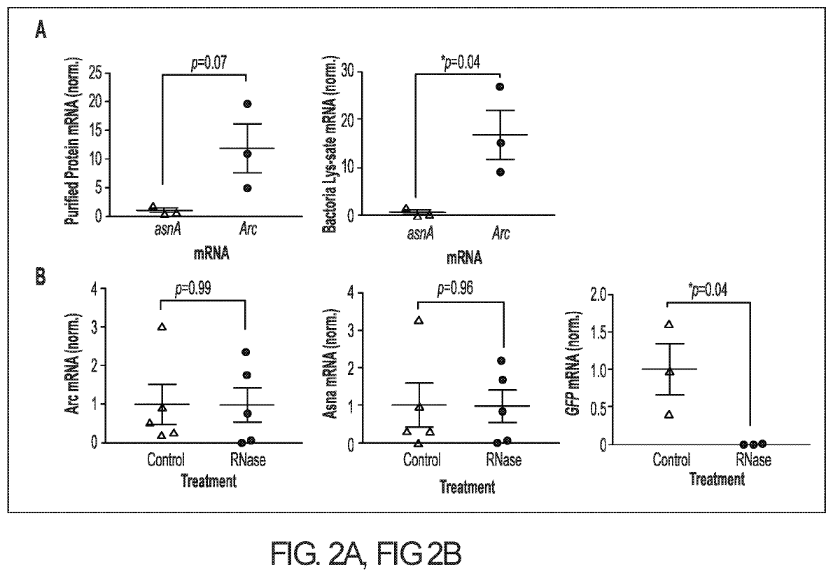

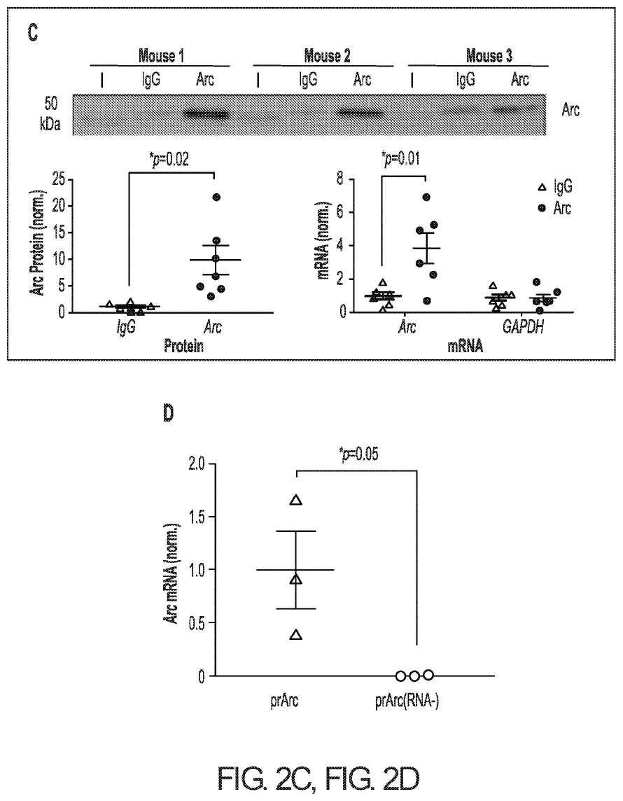

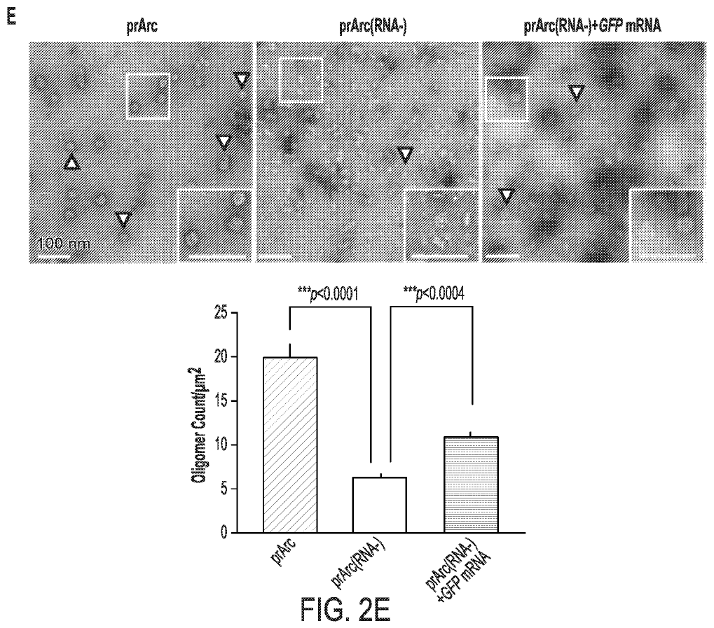

Retroviral encapsulation of viral genomic RNA is a complex process mediated by a network of interactions between Gag, RNA and lipid membranes (Mailler et al., 2016). HIV Gag contains zinc-finger knuckle motifs in the NC domain that mediate viral RNA binding and selection (Carlson et al., 2016), but in the absence of viral RNA, Gag can also bind cellular mRNAs, which may reflect nonspecific RNA interactions with the basic MA and NC domains (Comas-Garcia et al., 2016). Interestingly, Foamy Virus Gags do not contain zinc-finger domains and bind RNA through C-terminal glycine-arginine-rich patches (Hamann and Lindemann, 2016), suggesting that distinct Gag domains from different viral families have evolved to perform similar biochemical processes. Like Foamy Virus Gag, Arc does not appear to contain zinc-finger domains but may bind RNA through ionic interactions in its N terminus. prArc appeared to co-purify with RNA or other nucleic acids, as the preparations had a higher A260/280 spectrophotometric ratio than would be expected for a pure recombinant protein (prArc 1.04±0.024; Endophilin3A 0.55±0.006; n=3, p<0.01; FIG. 9B). The thought was that Arc can bind and encapsulate RNA. To ascertain whether prArc capsids contain mRNA, levels of Arc mRNA and a highly abundant bacterial mRNA, asnA (Zhou et al., 2011), were determined using qRT-PCR. Both Arc and asnA mRNA (FIG. 2A) were determined.

However, Arc mRNA levels were 10-fold higher than asnA. Bacterial cell lysate contained 15-fold higher Arc mRNA levels than asnA (FIG. 2A), indicating that prArc capsids show little specificity for a particular mRNA, but encapsulate abundant RNA according to stoichiometry. If mRNA is encapsulated in capsids, it should be resistant to ribonuclease (RNase) treatment. RNase did not degrade Arc or asnA mRNA, but significantly degraded exogenous free GFP mRNA (FIG. 2B), indicating that Arc and asnA mRNA were protected from RNase degradation.

iv. Whether Arc Protein Associates with Arc mRNA was Tested In Vivo by Immunoprecipitating Arc Protein from Mouse Cortical Lysate, Followed by qRT-PCR (FIG. 2C). Arc mRNA was Found to Selectively Immunoprecipitate (IP) with Arc Protein, while GAPDH was not Enriched in Arc IPs. These Results Indicate that Arc Protein and its mRNA Form a Complex in Neurons In Vivo.Arc Capsid Assembly Requires RNA

To form the immature viral capsid, HIV Gag must bind RNA (Mailler et al., 2016). To test whether Arc capsid formation requires RNA, full-length Arc protein was purified as above and then stripped bound nucleic acids (“prArc(RNA),” Figure S3A) as previously performed on HIV Gag (Ganser et al., 1999). This procedure reduced the A260/280 ratio significantly (prArc(RNA) 0.68±0.03, prArc 1.04±0.024; n=3, p<0.05) and Arc mRNA association was unable to be detected by qRT-PCR (FIG. 2D). Stripping RNA resulted in significantly fewer fully formed capsids (FIG. 2E), indicating that Arc capsids require RNA for normal assembly. To show directly that RNA facilitated Arc capsid assembly, GFP mRNA was exogenously added to prArc(RNA) (7.3% w/w), which resulted in significantly more fully formed Arc capsids.

v. Arc Protein and Arc mRNA are Released by Neurons in Extracellular Vesicles

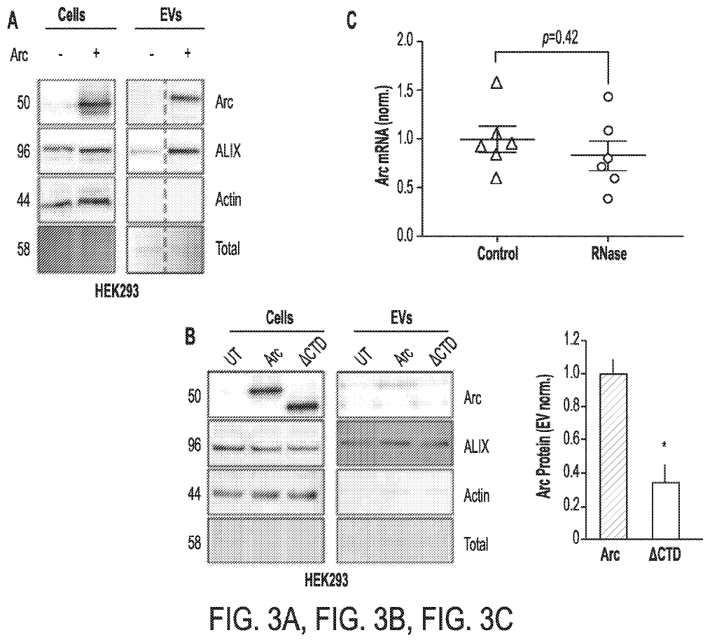

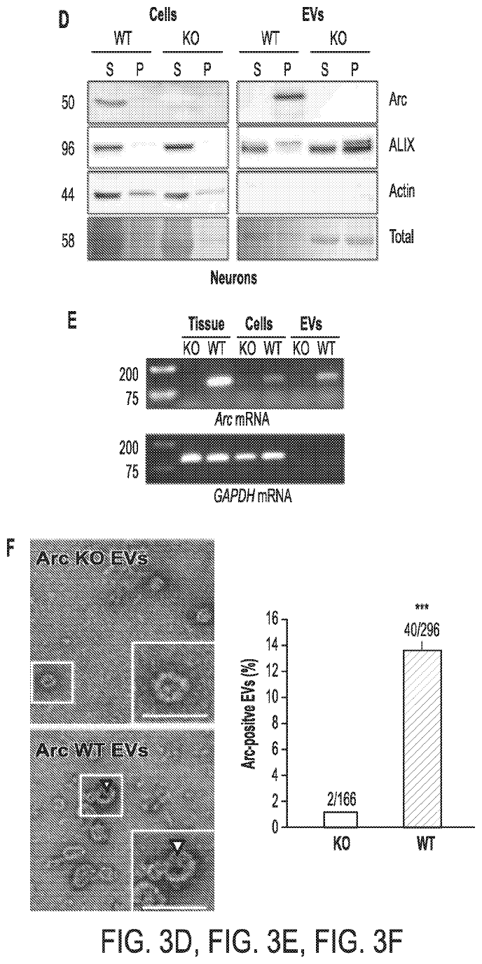

Retroviral capsids and EVs are released from cells using similar cellular machinery, such as the MVB pathway (Nolte't Hoen et al., 2016). Since Arc exhibits many of the biochemical properties of a viral Gag protein, whether Arc protein might also be released from cells was tested. Media was harvested from Arc-transfected HEK293 cells and the EV fraction was purified. This fraction contained vesicular structures that were <100 nm and resembled exosomes (Figure S3B). Arc protein was detected in the EV fraction, which was also positive for the EV marker ALIX, but lacked actin (FIG. 3A). Conversely, Arc-ΔCTD-transfected HEK cells exhibited little expression in the EV fraction (FIG. 3B), indicating that proper Arc capsid assembly can be required for Arc release via EVs. qRT-PCR was performed on the EV fraction from HEK cell media and detected Arc mRNA that was resistant to RNase treatment (FIG. 3C).

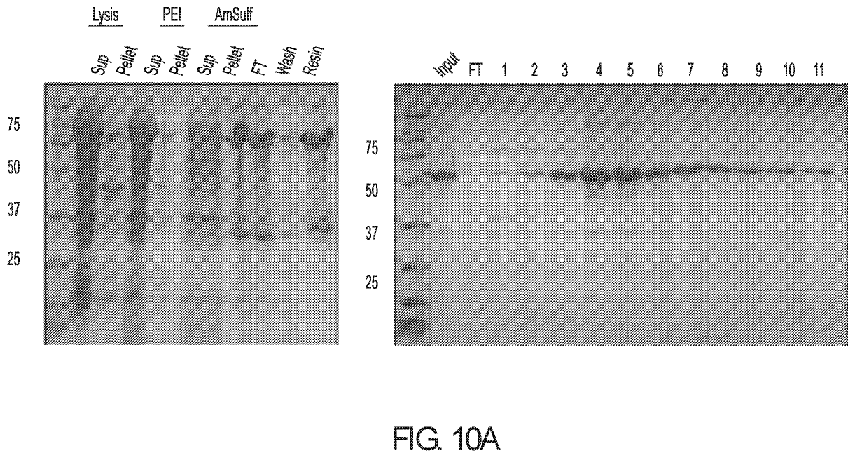

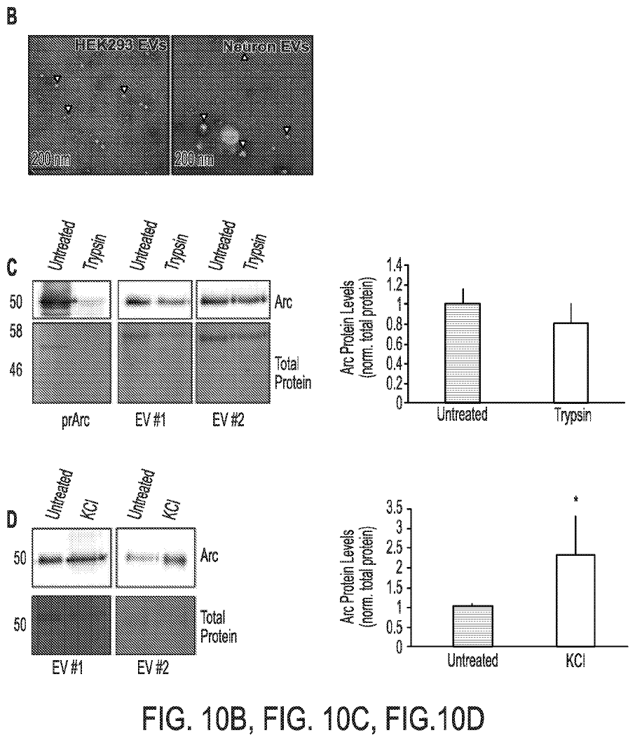

Native Arc protein was also found in the EV fraction prepared from media harvested from IV15 cultured cortical mouse neurons (FIG. 3D). Since Arc mRNA associates with Arc protein in brain lysate, RT-PCR was used to show that Arc mRNA is also present in EVs purified from neurons (FIG. 3E). Arc protein in EVs was resistant to trypsin digestion (FIG. 10C), indicating that Arc protein and RNA were protected or bound in a complex within EVs. To directly determine whether Arc protein is present in EVs, immunogold-labeling of endogenous Arc was conducted in the EV fraction from cultured neurons and found that Arc is present in a subpopulation of EVs (FIG. 3F). To test whether Arc release in EVs is activity dependent, the EV fraction was purified from media collected from untreated or KCl-treated wild-type (WT) cultured cortical neurons (FIG. 10D). KCl treatment, which increases neuronal activity, resulted in significantly more Arc released into the media.

vi. Arc Mediates Intercellular Transfer of its Own mRNA in Extracellular Vesicles

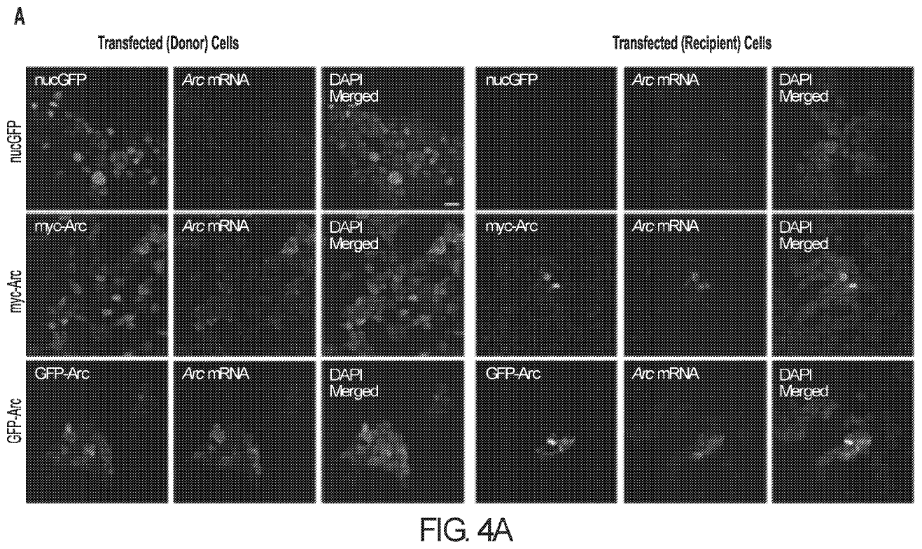

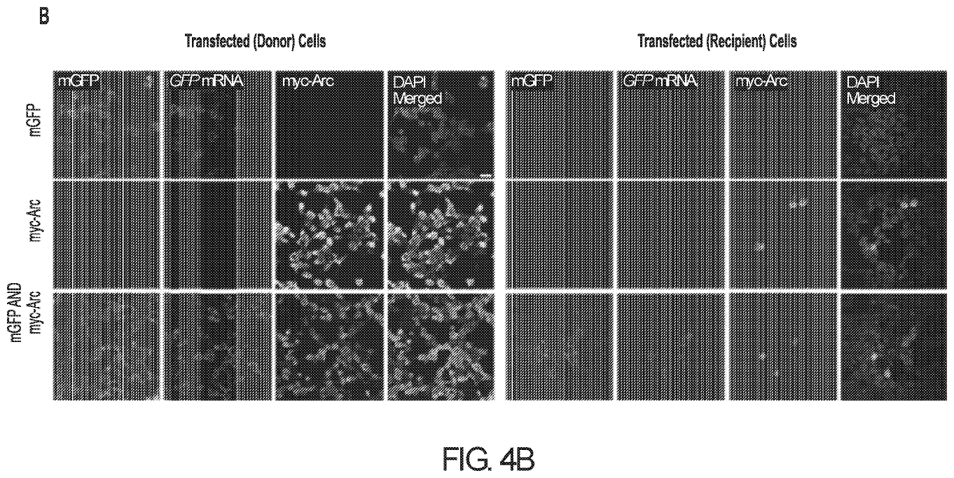

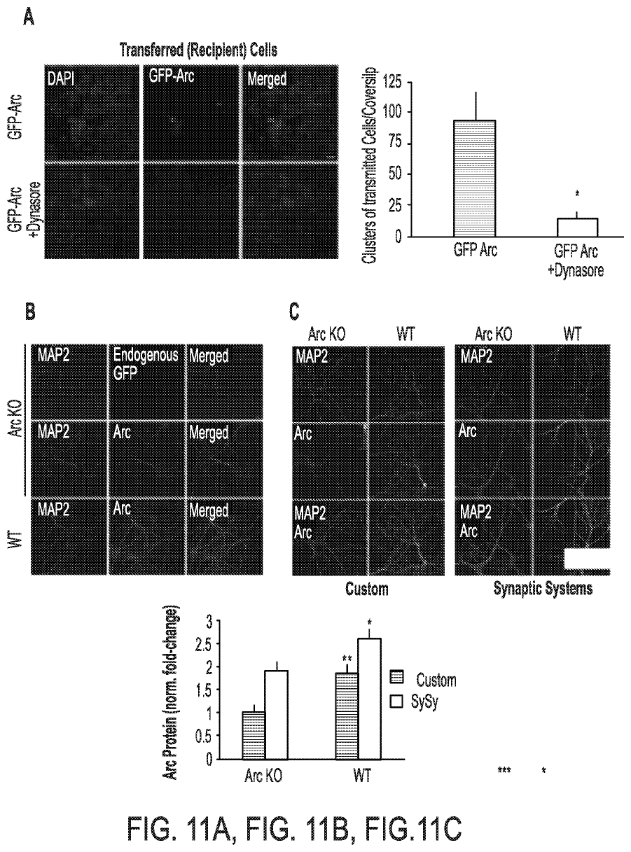

Virus particles are able to infect cells through complex interactions of the viral envelope and host cell membrane, while EVs can also transfer cargo such as RNAs cell-to-cell (Valadi et al., 2007). Arc can transfer mRNA, either directly via mRNA encapsulated in prArc or in Arc-containing EVs. GFP/myc-Arc or nuclear-GFP was transfected into HEK (donor) cells and media collected from these cells after 18 hr, which was then incubated with untransfected, naïve HEK (recipient/“transferred”) cells for 24 hr. High Arc expression was observed in a sparse population of naïve HEK cells (FIG. 4A), while cells incubated with media from cells transfected with nuclear-GFP alone did not express nuclear-GFP. Fluorescent in situ hybridization (FISH) for Arc mRNA revealed high levels of Arc mRNA in recipient cells. Uptake of Arc protein and mRNA was endocytosis-dependent, as application of Dy nasore (a potent inhibitor of clathrin-dependent endocytosis [Macia et al., 2006]) significantly blocked transfer of Arc protein (FIG. 11A). Since encapsulation of RNA by Arc capsids is nonspecific in vitro, whether Arc could co-transfer highly abundant mRNAs was tested. Donor HEK cells were transfected with myc-Arc and/or a membrane-bound GFP (mGFP), and media were collected after 24 hr. Recipient HEK cells showed clear transfer of both GFP protein and mRNA when donor cells contained Arc (FIG. 4B). No transfer was observed from cells transfected only with mGFP. These data indicate that Arc EVs released from HEK cells are capable of transferring highly abundant mRNAs cell-to-cell.

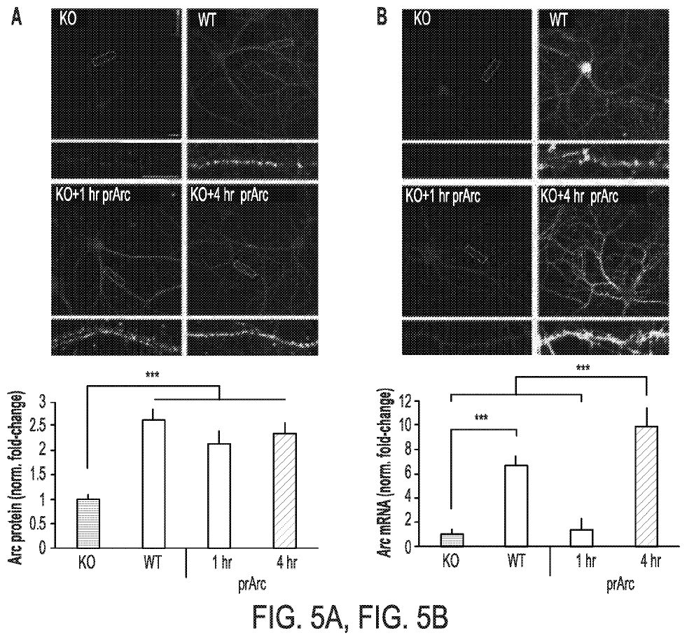

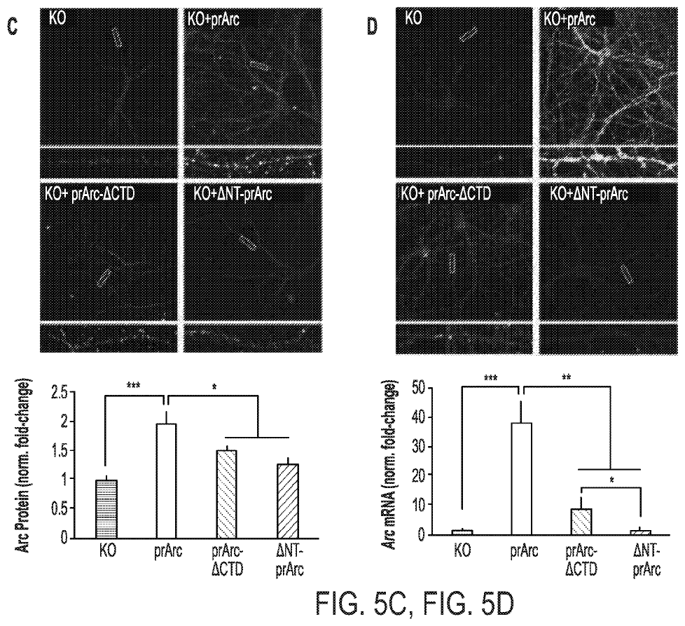

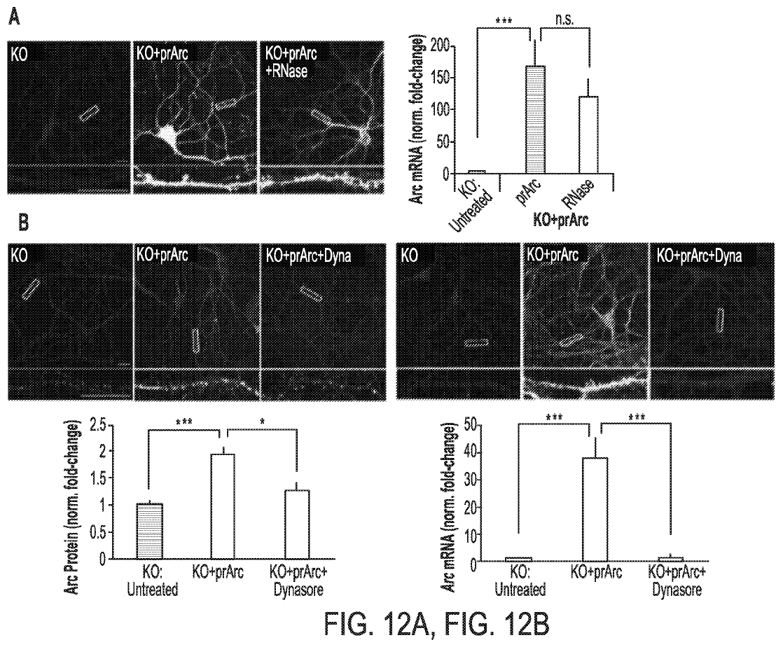

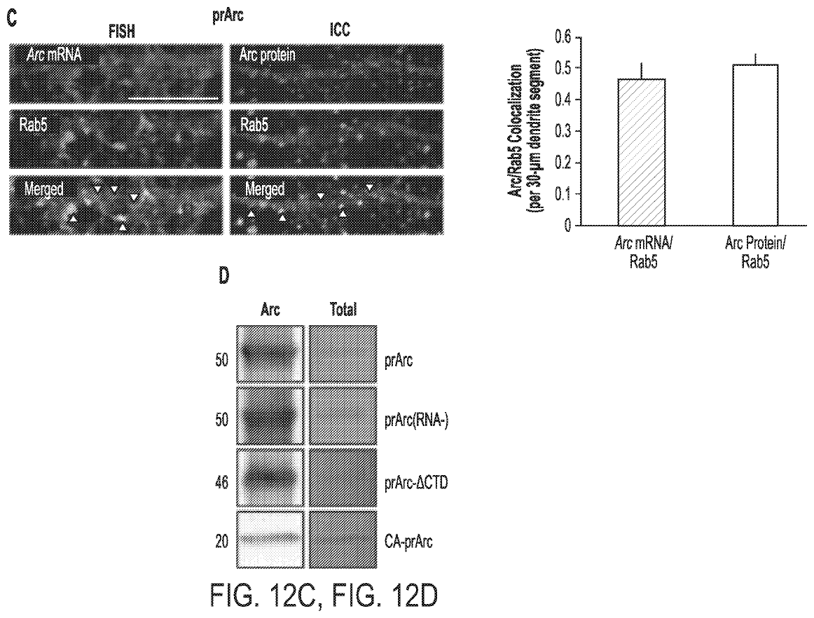

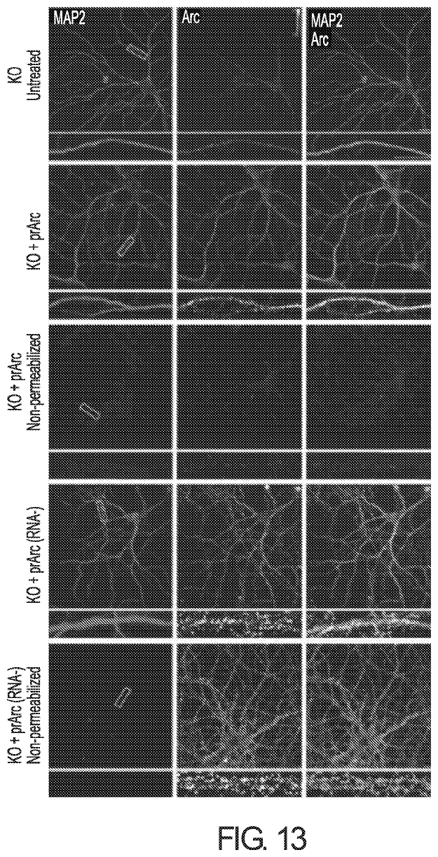

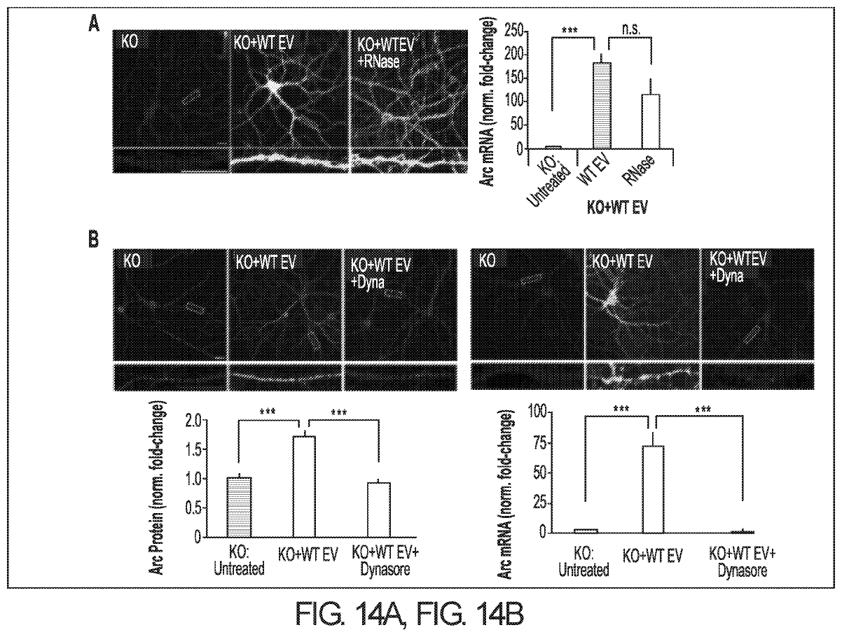

To test whether Arc capsids can transfer Arc mRNA into neurons, cultured hippocampal neurons from Arc knockout (KO) mice were incubated with prArc. Since the Arc KO line contains GFP knocked into the Arc locus (Wang et al., 2006), Arc was imaged in the red channel and were unable to detect GFP fluorescence in the green channel (FIG. 11B). Uptake of Arc protein into KO neurons was observed above antibody background levels (see FIG. 11C for antibody specificity) within 1 h of protein incubation, which peaked around 4 h of incubation (FIG. 5A). To directly determine whether Arc capsids can transfer Arc mRNA into neurons, Arc mRNA levels were measured in Arc KO neurons incubated with prArc. Arc FISH showed robust and high levels of transferred Arc mRNA after 4 h of incubation with prArc (FIG. 5B). RNase treatment of prArc prior to incubation had no effect on mRNA transfer (FIG. 12A), further indicating that Arc capsids are able to protect and encapsulate Arc mRNA. Blocking endocytosis using Dynasore prevented uptake of both prArc protein and Arc mRNA (FIG. 12B). Transferred mRNA and protein were evident both in early endosomes (marked by Rab5) and non-endosome compartments in dendrites (FIG. 12C). Both uptake and transfer of purified prArc-ΔCTD and CA-prArc protein and mRNA was significantly less than the full-length protein, indicating that capsid formation is required for uptake into neurons (FIGS. 5C and 5D). Lack of protein uptake was not due to poor detection by the custom-made Arc polyclonal antibody (FIG. 12D). Strikingly, prArc(RNA) was unable to be taken up but instead coated the outside of neurons (FIG. 13), further indicating that intact Arc capsids are required for uptake and transfer.

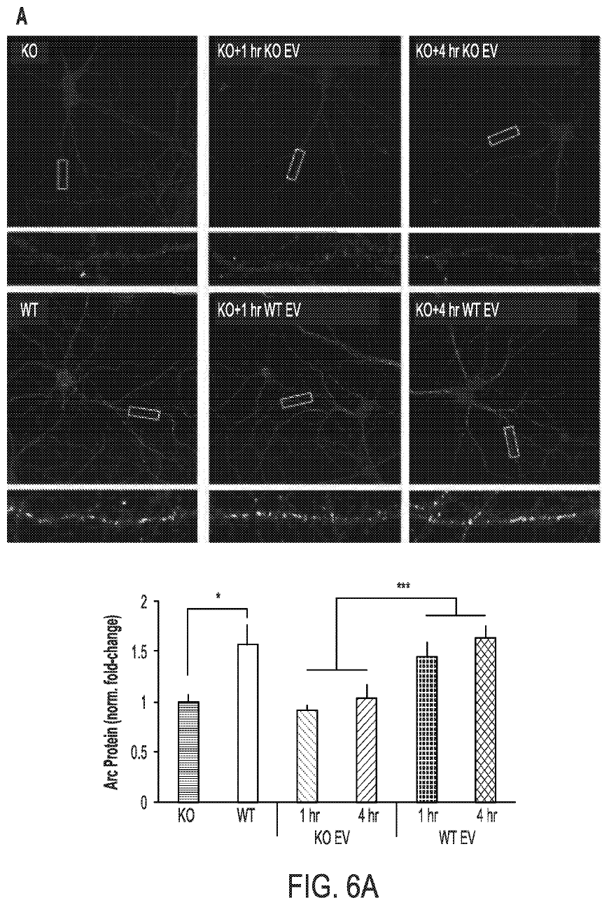

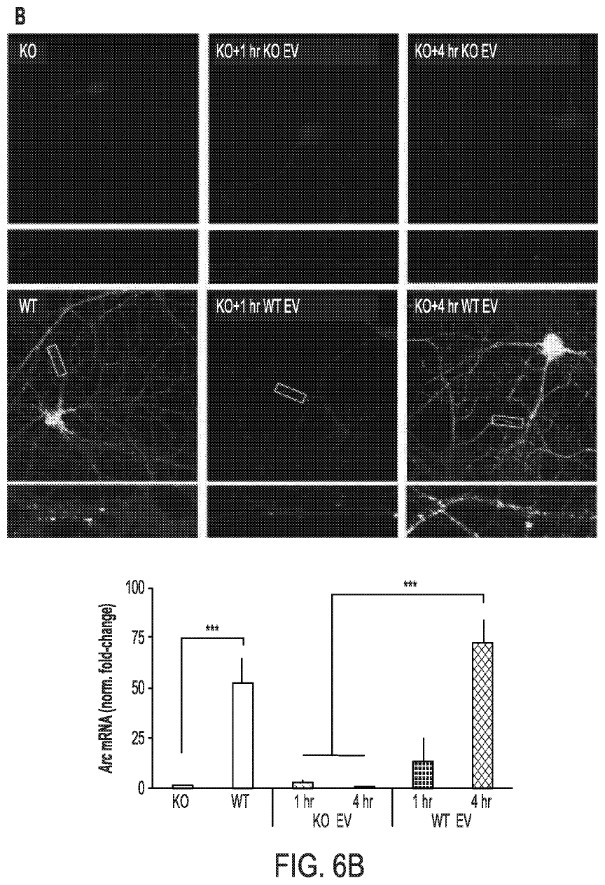

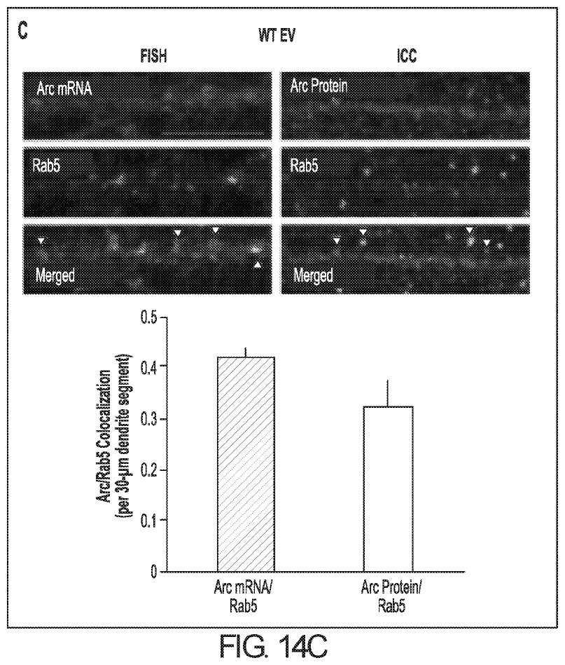

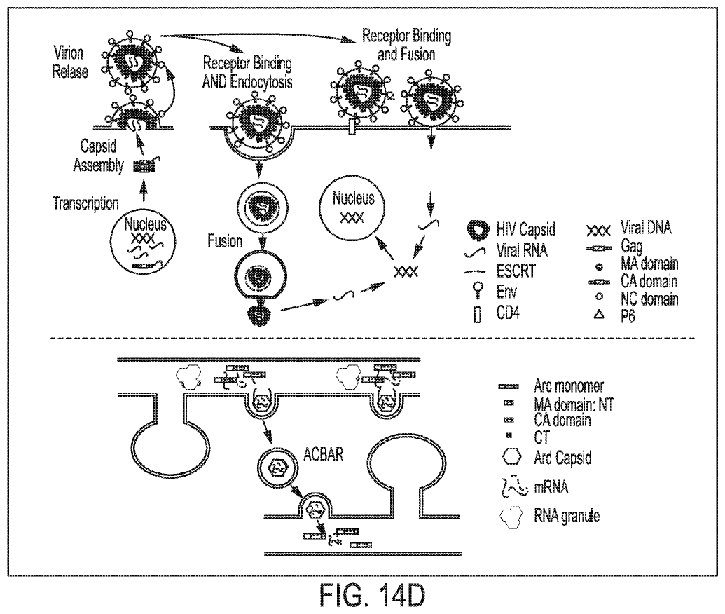

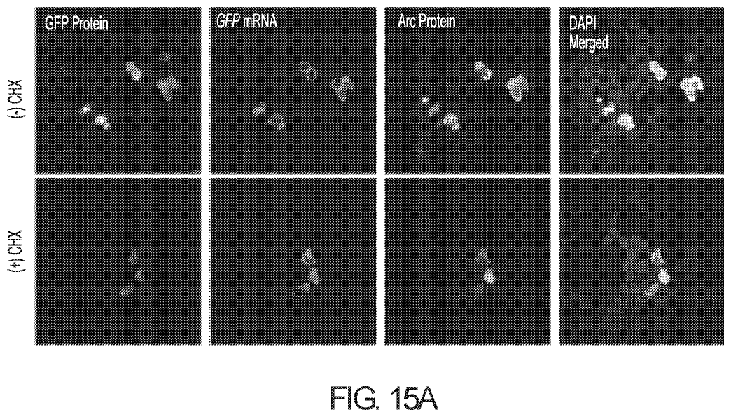

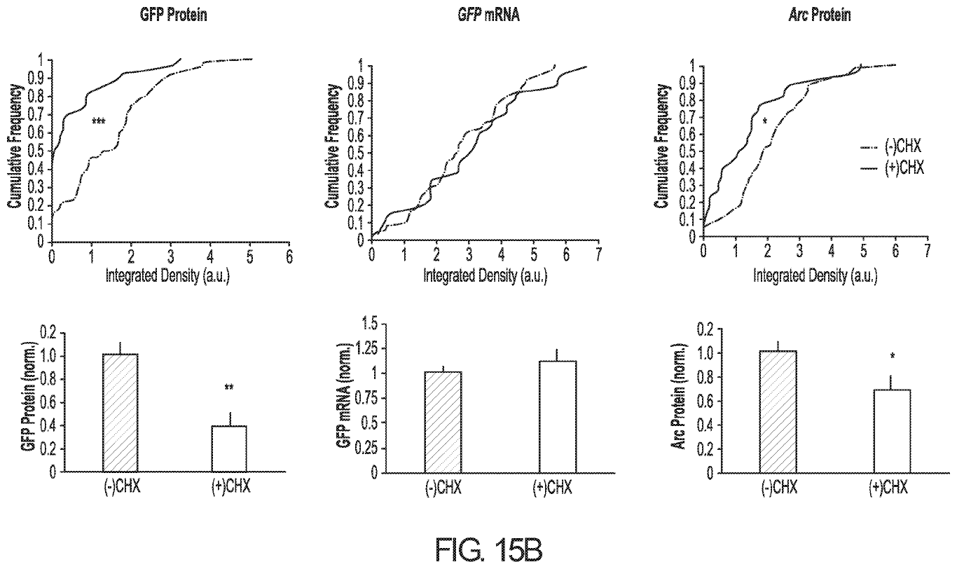

To test whether endogenous Arc can transfer mRNA, Arc KO cultured hippocampal neurons were incubated with purified EVs prepared from media from WT or KO cortical neurons. Arc KO neurons incubated with WT EVs showed a clear increase in dendritic Arc levels, while KO neurons incubated with EVs derived from KO cells exhibited no increase in dendritic Arc levels (FIG. 6A). In addition, FISH showed that Arc mRNA in WT EVs was transferred into KO neurons (FIG. 6B). Uptake of Arc mRNA was not significantly affected by prior treatment of EVs with RNase (FIG. 14A), indicating that uptake was not due to free or unbound Arc mRNA in the EV fraction. Blocking endocytosis with Dynasore prevented the uptake of Arc protein and mRNA from EVs (FIG. 14B). Notably, transferred Arc mRNA expression exhibited cell-wide localization in both early endosomes and non-endosome compartments (FIG. 14C) and was virtually indistinguishable from Arc mRNA distribution in WT neurons. These data indicate that endogenous Arc released via EVs is able to transfer Arc mRNA neuron-to-neuron.

vii. Transferred Arc mRNA can Undergo Activity-Dependent Translation

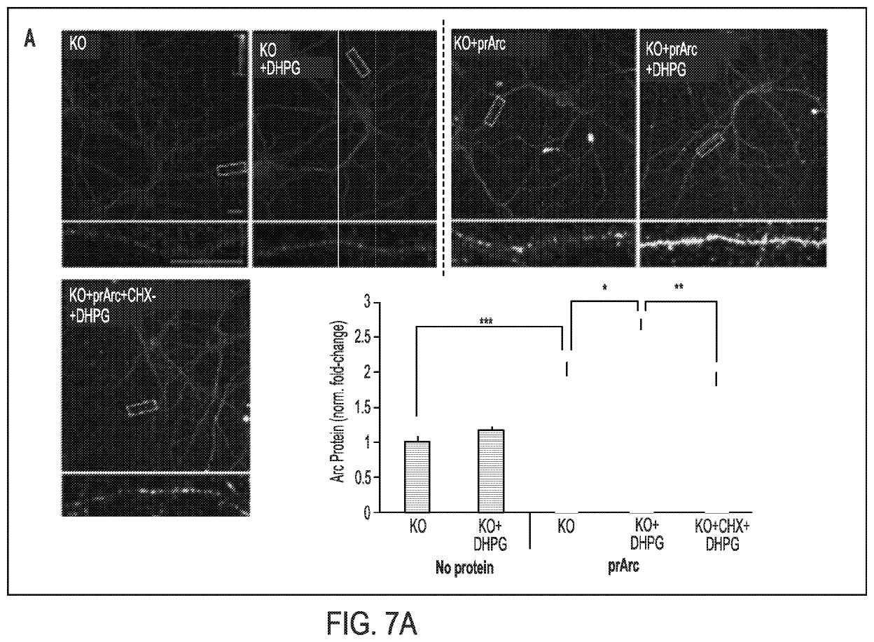

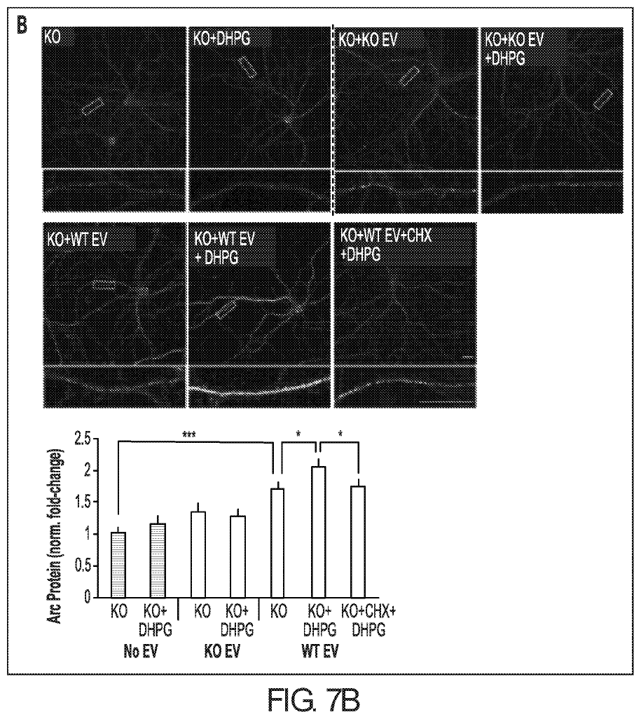

Arc mRNA associated with Arc capsids is transferred into the cytoplasm of neurons, an increase in dendritic Arc protein by inducing translation of Arc mRNA through activation of the group 1 metabotropic glutamate receptor (mGluR1/5) by the agonist DHPG, as previously shown for endogenous Arc (Waung et al., 2008) would be observed. As predicted, Arc protein levels were significantly increased in dendrites of Arc KO neurons after DHPG (5 min; 100 mM) application in cells incubated with prArc (FIG. 7A). This increase was not evident if a protein synthesis inhibitor (cycloheximide; 180 mM) was applied prior to DHPG application. KO neurons incubated with WT EVs for 4 hr and then treated with DHPG exhibited an increase in dendritic Arc levels that was also dependent on protein synthesis (FIG. 7B). Although these experiments cannot definitively distinguish de novo translated Arc from protein that was taken up, these data indicate that Arc capsids or EVs are capable of transferring Arc mRNA between neurons and that this mRNA is available in the cytoplasm of dendrites for activity-dependent translation

Mammalian Arc protein exhibits the main hallmarks of Gag proteins encoded by retroviruses and retrotransposons: self-assembly into capsids, RNA encapsulation, lipid binding, release in EVs, and intercellular transmission of RNA. These data indicate that Arc mediates intercellular trafficking of mRNA via Arc EVs (which we term ACBARs for Arc Capsids Bearing Arc RNA), revealing a novel molecular mechanism by which genetic information may be transferred between neurons.

viii. Arc Functions as a Repurposed Gag Protein

The data show a remarkable conservation of viral Gag properties in Arc. Since Arc shows structural homology to the Gag CA domain (Zhang et al., 2015), the capability of self-assembly into oligomeric capsids is perhaps not too surprising. However, Arc seems to retain other important biochemical properties of Gag that are not intuitive from its sequence. Despite lacking clear zinc-finger RNA binding domains such as in HIV Gag, Arc encapsulates RNA, and RNA binding seems critical for capsid formation. This is reminiscent of Foamy Virus Gags, which have evolved different RNA-binding motifs to HIV Gag (Hamann and Lindemann, 2016) and also structurally resemble Arc (Taylor et al., 2017). HIV Gag-RNA interactions are complex and involve multiple components of Gag, including the MA domain, and are regulated by host cellular factors (Mailler et al., 2016). Gag MA-RNA interactions are also critical for virus particle formation at membranes (Kutluay et al., 2014). Moreover, if viral RNA is not present, Gag encapsulates host RNA, and any single-stranded nucleic acid longer than 20-30 nt can support capsid assembly (Campbell and Rein, 1999), indicating a general propensity to bind abundant RNA. Indeed, precisely how viral RNA is preferentially packaged into Gag capsids in cells remains an intensive area of investigation (Comas-Garcia et al., 2016).

The uptake and transfer of RNA by purified Arc protein is surprising as this occurs in the absence of an “envelope” or lipid bilayer. Uptake of both purified Arc capsids and endogenous EVs occurs through endocytosis. While EVs and exosomes are easily taken up through the endosomal pathway, it remains unclear how RNA can cross the endosomal membrane without membrane fusion proteins (Tkach and Théry, 2016). The data indicate that, like non-enveloped viruses, Arc protein itself contains the ability to transfer RNA across the endosomal membrane. While it remains unclear how non-enveloped capsids transfer RNA into the cytoplasm, some studies suggest this could occur through specific receptor-capsid interactions, or via a pH-dependent conformational change of the capsid that allows either pore formation or lytic degradation of membranes (Tsai, 2007). Arc protein can interact with the endosomal membrane to allow transfer of mRNA into the cytoplasm as the capsid is disassembled. This is reflected in the lag between protein uptake and mRNA expression seen in these experiments, which can be a result of the time it takes for mRNA to become accessible to our FISH probes. The lipid membrane around ACBARs in vivo may dictate targeting and uptake, whereas the Arc capsid within protects and allows transfer of RNA. Intriguingly, prArc that lacks RNA is unable to form capsids and cannot be taken up, indicating uptake can be a regulated process that requires properly formed capsids. Since Arc seems to regulate a naturally occurring mechanism of RNA transfer, harnessing this pathway can allow new means of genetic engineering or RNA delivery into cells, using ACBARs, that can avoid the hurdle of immune activation.

ix. Arc's Gag Homology Reveals a New Signaling Pathway in Neurons

Exosome and EV signaling has emerged as a critical mechanism of intercellular communication, especially in the immune system and in cancer biology (Becker et al., 2016). However, the role of intercellular signaling through EVs in the nervous system has only recently been investigated, with studies suggesting that these pathways may play important roles in synaptic plasticity (Budnik et al., 2016; Zappulli et al., 2016). Canonical exosomes are formed in MVBs, which are derived from the endosomal pathway and usually require the ESCRT complex to be released (Raposo and Stoorvogel, 2013), although the biogenesis of EVs in general is more varied. HIV Gag is able to form virions independent of the MVB pathway, although the ESCRT machinery is still required for particle release; thus, Arc may form ACBARs independent of the canonical exosome pathway. These pathways are not mutually exclusive, and elucidating the biogenesis of ACBARs within neurons will require further investigation.

Since Arc is rapidly synthesized locally in dendrites (Park et al., 2008; Waung et al., 2008), it is conceivable that high local concentrations of Arc protein promote capsid assembly in dendrites where encapsulation of dendritically localized mRNAs could occur. Since Arc capsids do not seem to show specificity in RNA binding in vitro and Arc EVs can transfer highly abundant mRNAs, we speculate that the specificity of ACBAR cargo is conferred by the precise spatial and temporal expression of Arc protein in neurons (FIG. 14D). Consistent with the identification of Arc mRNA associated with Arc protein from brain, Arc mRNA levels are highly and uniquely abundant in dendrites in vivo after bouts of neuronal activity or experience (de Solis et al., 2017). Gag-RNA interactions are regulated by host cellular proteins such as Staufen (Mouland et al., 2000), a protein that is also a critical regulator of dendritic mRNA trafficking in neurons, including Arc mRNA (Heraud-Farlow and Kiebler, 2014). The parallels between dendritic mRNA regulation and virus-RNA interactions are striking, indicating that cellular factors can play an important role in ACBAR biogenesis and RNA packing.

The data also indicates that Arc can mediate intercellular signaling to control synaptic function and plasticity in a non-cell-autonomous manner. Although there is a paucity of data on neuronal EVs, previous studies have shown that EVs can be secreted in an activity-dependent manner and include AMPARs as cargo (Faure et al., 2006). Since Arc has previously been implicated in AMPAR trafficking at synapses and spine elimination (Chowdhury et al., 2006; Mikuni et al., 2013) at weak synapses (Okuno et al., 2012), a potential role for ACBARs may be to eliminate synaptic material. Arc also regulates homeostatic forms of plasticity, such as AMPAR scaling (Shepherd et al., 2006) and cross-modal plasticity across different brain regions (Kraft et al., 2017), which could be regulated at the circuit level in a non-cell autonomous manner. Released Arc functions to carry intercellular cargo that alters the state of neighboring cells required for cellular consolidation of information.

Previous studies have shown that Drosophila neuromuscular junction plasticity requires trans-synaptic signaling mediated through the Wnt pathway in exosomes (Korkut et al., 2009). Interestingly, the Drosophila Arc homolog dArc1 exhibits similar properties of intercellular transfer of mRNA in the fly nervous system and is one of the most abundant proteins in Drosophila EVs (Ashley et al., 2018; Lefebvre et al., 2016), indicating a remarkable convergence of biology despite a large evolutionary divergence of these species. A recent study has also implicated Arc in the mammalian immune system (Ufer et al., 2016), where it controls dendritic cell-dependent T cell activation, expanding the potential repertoire and importance of Arc-dependent intercellular signaling beyond the nervous system. Moreover, EVs have been implicated in the pathology of various neurodegenerative disorders, as several pathogenic proteins, such as prions, β-amyloid peptide, and α-synuclein, are released from cells in association with EVs (Zappulli et al., 2016). In AD, immunohistochemical analysis in brain sections from patients with AD showed enrichment of the exosomal marker ALIX around neuritic plaques (Rajendran et al., 2006). This suggests that EVs may provide a significant source of extracellular AP peptide. Arc regulates the activity-dependent cleavage of APP and b-amyloid production through interactions with presenilin (Wu et al., 2011), indicating that ACBARs can also be involved in AD pathogenesis.

x. Evolution of Synaptic Plasticity and Cognition

Ty3/gypsy retrotransposons are ancient mobile elements that are widely distributed and often abundant in eukaryotic genomes and are considered ancestral to modern retroviruses (Malik et al., 2000). There is evidence that coding sequences derived from Ty3/gypsy and other retroviral-like elements have been repurposed for cellular functions repeatedly during evolution (Feschotte and Gilbert, 2012). For instance, multiple envelope genes of retroviral origins have been co-opted during mammalian evolution to promote cell-cell fusion and syncytiotrophoblast formation in the developing placenta (Cornelis et al., 2015). There are more than one hundred Gag-derived genes in the human genome alone (Campillos et al., 2006), and genetic KOs of their murine orthologs have revealed that some, like Arc, are essential for cognition (Irie et al., 2015). However, the molecular function of these Gag-derived proteins has been poorly characterized, and whether they were co-opted to serve similar cellular processes remains an open question. This study and the accompanying article from Ashley et al. (2018) now reveal that two distantly related Gag-derived genes have been independently co-opted in fly and tetrapod ancestors to participate in a similar process of EV-dependent intercellular trafficking of RNA in the nervous system.

2. Methods

i. Plasmids

The open reading frame (ORF) of full-length rat Arc (NP_062234.1) cDNA was subcloned from pRK5-myc-Arc. The insert was amplified by PCR, digested with BamH1 and Xho1, and ligated into the pGEX-6p1 (GE Healthcare, Little Chalfont, UK) expression vector between the BamH1 and Xho1 restrictions sites. The GST-Arc ORF was similarly amplified and cloned into the pFastBacl vector (Thermo Fisher Scientific) between the BamH1 and Xho1 restriction sites. prArc-ΔCTD was generated by blunt end cloning after PCR amplification of the Arc ORF from pGEX-6p1-Arc, excluding sequence coding aas 277-374. aas 195-364 of the Arc ORF (CA-prArc) was similarly cloned into the pET11 a vector, which contained a His tag. pBluescript-SKII-GFP was generated by restriction digest of mEGFP (BBA16881.1) from pGL4.11-arc7000-mEGFP-ArcUTRs (generously provided by Dr. Haruhiko Bito, University of Tokyo) and subsequent ligation into the KpnI and SacI restriction sites flanking the insert in pBluescript-SKII-ArcUTRs plasmid (generously provided by Dr. Kristen Keefe, University of Utah). The pGEX-4T-1 Drosophila Arc1 (NP_610955.1) construct was provided by Dr. Mark Metzstein, University of Utah. EGFP-C3-Arc and pRK5-myc-Arc were generously provided by Dr. Kimberly Huber (UT Southwestern) and Dr. Paul Worley (Johns Hopkins University), respectively. All protein expression constructs were transformed into DH5 a E. coli cells and individual colonies were screened by Sanger Sequencing (GeneWiz, South Plainfield, N.J.) sequencing services, using primers synthesized by Integrated DNA Technologies (Coralville, Iowa). Trace files were analyzed using A Plasmid Editor (APE) freeware available from the University of Utah. Sequenced verified constructs were then transformed into BL21-DE3 bacterial cells for protein expression.

ii. Protein Purification

Starter bacteria cultures for protein expression were grown overnight at 37° C. in LB supplemented with ampicillin and chloramphenicol. Starter cultures were used to inoculate large-scale 500 mL cultures of ZY auto-induction media. Large-scale cultures were grown to OD600 of 0.6-0.8 at 37° C. at 150 rpm and then shifted to 19° C. at 150 rpm for 16-20 h. Cultures were then pelleted at 5000×g for 15 min at 4° C. and cell pellets were resuspended in 30 mL lysis buffer (500 mM NaCl, 50 mM Tris, 5% glycerol, 1 mM DTT, pH 8.0 at room temperature (RT) for Arc constructs and GST; 300 mM KCl, 50 mM Tris, 1% Triton X-100, 1 mM DTT, pH 7.4 at RT for Endophilin3A) and flash frozen in liquid nitrogen. Frozen pellets were thawed quickly at 37° C. and brought to a final volume of 1 g pellet:10 mL lysis buffer, supplemented with DNase, lysozyme, aprotinin, leupeptin, PMSF, and pepstatin. Lysates were then sonicated for 8-10×45 s pulses at 90% duty cycle and pelleted for 45 min at 21,000×g. For GST-tagged constructs, cleared supernatants were then passed through a 0.45 mm filter and incubated with pre-equilibrated GST Sepharose 4B affinity resin in a gravity flow column overnight at 4° C. Bound protein was then washed twice with two column volumes (20 resin bed volumes each) of lysis buffer, re-equilibrated with 150 mM NaCl, 50 mM Tris, 1 mM EDTA, 1 mM DTT, pH 7.2 at RT, and cleaved on-resin overnight at 4° C. with PreScission Protease (GE Healthcare) for the GST-tagged constructs, or thrombin (Sigma-Aldrich) for dArc1. Cleaved proteins were then buffer exchanged to 150 mM NaCl, 50 mM Tris, pH 7.4 at RT to kill protease activity, run on an S200 size exclusion column to separate the cleaved protein, and peak fractions were pooled. GST was affinity-purified as described above using Sepharose 4B resin and eluted directly using 15 mM reduced L-glutathione, 10 mM Tris, pH 7.4 at RT. His-tagged CA-prArc was affinity-purified as described above using Ni+ resin (Roche, Basel, Switzerland) and eluted directly using 250 mM imidazole, 10 mM Tris, pH 7.4 at RT. GST and CA-prArc were then buffer exchanged to 150 mM NaCl, 50 mM Tris, pH 7.4 at RT. To strip Arc protein of nucleic acids for prArc(RNA−) preparations, cell pellets were lysed in 20 mM NaCl, 50 mM Tris, 5% glycerol, 2 mM MgCl2, 1 mM DTT, pH 8.0 at RT as described above. Nucleic acids were precipitated from cell supernatants by dropwise addition of 10% PEI, pH 8.0 to a final concentration of 0.1% followed by incubation at 4° C. for 20 min and pelleting for 20 min at 27,000×g. The resulting supernatant was then precipitated by addition of saturated ammonium sulfate to a final concentration of 30%. Precipitated protein was pelleted at 10,000×g for 10 min, resuspended in 60 mL lysis buffer, and affinity purified. The cleaved affinity-purified product was then dialyzed to Q-column buffer A (Q-A; 20 mM NaCl, 50 mM Tris, pH 7.4 at RT) overnight. Dialyzed protein was then subjected to anion exchange chromatography (HiTrap Q, GE Healthcare) with a gradient of Q-A buffer to Q-B buffer (1 M NaCl, 50 mM Tris, pH 7.4). Average yields for purified proteins were 10.5 mg (8-13 mg) per liter of cell culture. Electron microscopy

iii. Negative Stain

For all negative stain specimens, copper 200-mesh grids coated with Formvar and carbon (Electron Microscopy Sciences or TedPella, Redding, Calif.) were glow discharged for 20-45 s in a vacuum chamber at 30 mA. 3.5 mL sample was then applied to the grid for 35-45 s and excess sample was wicked away using filter paper. Grids were then immediately washed 2-4× for 5 s with 30 mL water droplets, then once with 1% uranyl acetate (UA) on parafilm. Excess water/UA was wicked away and then a final droplet of UA was applied for 30 s. Excess UA was wicked away and grids were air-dried for 30-60 s. Imaging was performed using either an FEI T12, FEI Tecnai Spirit microscope operated at 120 kV equipped with a Gatan Onus SC200B CCD camera or JEOL 1400 electron microscope.

a. Cryo-EM

Purified Arc protein was dialysed into 300 mM NaCl, 50 mM Tris, pH 7.4 and concentrated twice using Amicon 100 MWCO centrifugal filters (Millipore, Burlington, Mass.) to yield a final protein concentration of 2 mg/mL. 10 nm diameter gold beads were added to the sample. Degassed 2/2-3C C-flat grids (Electron Microscopy Sciences, Hatfield, Pa.) were glow discharged for 45 s at 30 mA. Sample was applied to the grid 2 times for 30 s, and the grid was plunge frozen in liquid ethane using a FEI Vitrobot Mark IV. Micrographs were acquired using a FEI Tecnai G2 F20 microscope operated at 200 kV, equipped with a FEI Falcon II direct detector. The nominal defocus was 1.3 mm.

b. EM Quantification

Grids were surveyed visually to check for uniformity of sample application. For each experiment, six images were taken from randomly selected grid squares. Full and partially formed particles between 20-40 nm were then counted manually using ImageJ. Counts were then divided by the image field of view (2.07 mm2) and data presented as oligomer count/mm2.

c. Arc Capsid Assembly Assay

GFP mRNA was added to prArc(RNA−) (5 mg/mL in low salt buffer: 20 mM NaCl, 50 mM Tris, pH 7.4 at RT) at a nucleic acid:protein ratio of 7.3% (w/w) (corresponding to 1 molecule of Arc to 10 nucleotides). Reactions were then diluted to 1 mg/mL of prArc(RNA−) by dropwise addition of low salt buffer or capsid assembly buffer (500 mM NaPO4, 50 mM Tris, 0.5 mM EDTA, pH 7.5 at RT) and incubated for 2 h at RT. Following incubation, negative stain EM grids were prepared of each reaction at 0.25 mg/mL and capsid formation was quantified by manual counting of 6 images. Fully formed capsids included spherical particles between 20-50 nm with clear double shells. Similar results were seen in three independent protein preparations.

d. Dynamic Light Scattering

PurifiedArc protein was subjected to dynamic light scattering measurements on a Malvern Zetasizer Nano ZSP instrument. The scattering was carried out at 25° C. and at a fixed angle of 173 (backward scattering). The scattered intensity is represented as number of particles under the assumption that the scattering intensity from spherical particles is proportional to the size to the sixth power. Phylogenetic reconstruction e. Animals

NCBI genome sequence databases were queried using the human or Drosophila melanogaster Arc protein sequence using tBLASTn. Repbase was also queried using the CENSOR program to identify known repeat families with high sequence similarity to mammalian or brachyceran Arc genes, respectively. The following sequence IDs were used for analysis: (GenBank locus) Mm ARC—AHBB01089569; Hs ARC—LIQK02016549; Ac ARC—AAWZ02020354; Lc gypsy2—AFYHO1030203; CC gypsy—LHQP01046008; Dm ARC1—JSAE01000572; Ds ARC1—CAKG01020471; Sc ARC1—LDNW01019671; Dm ARC2—JXOZ01003752; Ds ARC2—AWUT01001000; Sc ARC2—LDNW01019670; Bm gypsy—BABH01046987; Tc gypsy—AAJJ02003810. Repbase: Lc gypsy—Gypsy2-1-I_Lch; Dr gypsy26—Gypsy-26-I_DR; Lh gypsy11—Gypsy-11_LH-I; Dm gypsy1—Gypsy1-I_DM; ty3—TY3. Protein (Arc and Gag) sequences that were found to have high similarity to Arc proteins and Gags of other related Ty3/gypsy elements were aligned using the MUSCLE program. Trimmed Arc/Gag alignments were uploaded to MEGA7 for subsequent maximum likelihood phylogenetic reconstruction using default parameters, and 500 bootstrap iterations were performed to generate a lineage tree. Drosophila melanogaster dArc1 and dArc2 protein sequences were used to query schizophoran fly protein databases using BLASTp. More hits were observed than expected if darc1 were present in one-to-one orthologs in the species examined. Protein FASTA sequences were aligned using MUSCLE and a maximum likelihood phylogram was generated using MEGA. Animals

Arc knock-out (KO) mice (a kind gift from Dr. Kuan Wang, NIH), which have GFP knocked in to the Arc ORF (Wang et al., 2006), and wild-type (WT) C57BL/6 littermates were used for hippocampal and cortical lysate experiments. Hippocampal and cortical primary neuronal cultures were prepared from WT or KO E18 embryos.

iv. Cell Culture

Primary neuron cultures were prepared from E18 mouse cortex and hippocampus as previously described (Shepherd et al., 2006). Tissue was dissociated in DNase (0.01%; Sigma-Aldrich) and papain (0.067%; Worthington Biochemicals, Lakewood, N.J.), and then triturated with a fire-polished glass pipette to obtain a single-cell suspension. Cells were pelleted at 1000×g for 4 min, the supernatant removed, and cells resuspended and counted with a TC-20 cell counter (Bio-Rad, Hercules, Calif.). Neurons were plated on glass coverslips (Carolina Biological Supply, Burlington, N.C.) coated with poly-L-lysine (0.2 mg/mL; Sigma-Aldrich) in 12-well plates (Greiner Bio-One, Monroe, N.C.) at 90,000 cells/mL, or in 10-cm plastic dishes at 800,000 cells/mL. Neurons were initially plated in Neurobasal media containing 5% horse serum, 2% GlutaMAX, 2% B-27, and 1% penicillin/streptomycin (Thermo Fisher Scientific) in a 37° C. incubator with 5% CO2. On DIV4, neurons were fed via half media exchange with astrocyte-conditioned Neurobasal media containing 1% horse serum, GlutaMAX, and penicillin/streptomycin, 2% B-27, and 5 μM cytosine □-D-arabinofuranoside (AraC) (Sigma-Aldrich). Neurons were fed with astrocyte-conditioned media every three days thereafter. HEK293 cells were maintained in DMEM media supplemented with 10% fetal bovine serum and 1% penicillin/streptomycin (Thermo Fisher Scientific) and passaged every 3-4 days at 70% confluency. For transfections and transfer experiments, HEK cells were seeded to 10-cm dishes or collagen-coated glass coverslips in 12-well plates.

v. Transfections

HEK cells were transfected using polyethyleneimine (PEI) at a ratio of 3 mg PEI:1 mg DNA diluted in Opti-MEM (Thermo Fisher Scientific). Cells were transfected at approximately 60%-70% confluency. For EV isolation and media transfer experiments, culture media was exchanged 4-6 h post-transfection to remove PEI and DNA, and media was harvested 24 h later. HEK cell transfer experiments

vi. Transfection and Transfer

Media from transfected HEK cells was harvested 24 h post-transfection and centrifuged at 500×g for 4 min to remove dead cells and debris. Media from untransfected, naive cells was removed and replaced with the cleared transfected media and incubated for an additional 24 h. Following incubation, cells were fixed and combined immunocytochemistry/fluorescence in situ hybridization (ICC/FISH) for Arc or GFP protein and RNA was performed as described below.

a. Endocytosis Blockade

To block endocytosis, a group of naive HEK cells plated on coverslips in 12-well plates that were receiving media from GFP-Arc-transfected HEK cells were treated at the same time with 80 mM Dynasore (Abcam, Cambridge, Mass.) for the first 6 h, then the media was removed and replaced with fresh HEK media. 18 h later, Dynasore-treated and untreated HEK cells were fixed. The entire 18-mm coverslip was viewed with a 20× objective and the number of clusters of GFP-Arc-transferred cells was manually counted. Representative images were obtained using a 20× objective on an Olympus FV1000 confocal microscope (Tokyo, Japan).

vii. Neuron Transfer Experiments

a. Imaging

DIV15 cultured neurons were used for all neuronal experiments. For purified Arc protein incubation experiments, neurons were treated with 4 mg of purified prArc, prArc-ΔCTD, CA-prArc, or prArc(RNA−) protein in normal neuronal feeding media and incubated for 1 or 4 h. For extracellular vesicle (EV) incubation experiments, neurons were treated with 10 mg protein from the purified EV fraction obtained from eight 10-cm dishes of DIV15 cultured cortical neurons in which E18 WT cortical neurons had been plated at 800,000 cells/mL (see “Cell Culture” methods), and incubated for 1 or 4 h. A subset of neurons in the purified protein- and EV-treated experiments was treated with 100 mM of the group 1 mGluR agonist dihydroxyphenylglycine ((S)-3,5-DHPG; Tocris Bioscience, Bristol, UK) for 5 min, which was then washed out and replaced with previously conditioned neuronal media, and neurons were allowed to rest for 25 min before fixation. To block protein translation during DHPG treatment, a subset of neurons was pretreated with 180 mM cycloheximide (CHX, Sigma-Aldrich) 30 min before DHPG. CHX was left in the media for 1 h total. To block endocytosis, neurons were pretreated with 80 mM Dynasore (Abcam, Cambridge, Mass.) for 30 min before adding purified protein. For RNase treatments, a sample of either prArc or WT EV was incubated with RNase A (1:1000; Omega Bio-tek, Norcross, Ga.) for 15 min, then SUPERase-In RNase Inhibitor (1 U/mL; Thermo Fisher Scientific) immediately before being added to neurons. The treated samples were then added to neurons and incubated for 4 h.

After treatments, neurons were washed twice with 37° C. 4% sucrose/1× phosphate-buffered-saline (PBS; 10×: 1.4 M NaCl, 26.8 mMKCl, 62 mM Na2HPO4, 35.3 mM KH2PO4, pH 7.4), then fixed for 15 min with 4% sucrose/4% formaldehyde (Thermo Fisher Scientific) in 1×PBS. Neurons were washed 335 min with 1×PBS, permeabilized for 10 min with 0.2% Triton X-100 (Amresco, Solon, Ohio) in 1×PBS, and blocked for 30 min in 5% normal donkey serum (Jackson ImmunoResearch, West Grove, Pa.) in 1×PBS. Neurons were then incubated in primary antibody diluted in block for 1 h at RT, washed 335 min in 1×PBS, and incubated in secondary antibody diluted in block for 1 h at RT. Neurons on coverslips were mounted on glass slides in Fluoromount (Thermo Fisher Scientific) and dried overnight at RT. Primary antibodies used were: rabbit anti-Arc (1:1000; custom-made; ProteinTech, Rosemont, Ill.); rabbit anti-Arc (1:1000; Synaptic Systems, Goettingen, Germany); chicken anti-MAP2 (1:5000; ab5392; Abcam); mouse anti-Rab5 (1:1000; BD Biosciences, San Jose, Calif.); DAPI nuclear stain (Molecular Probes, Thermo Fisher Scientific). Secondary antibodies used were: Alexa Fluor 405, 488, 555, or 647 for the appropriate animal host (1:750; Thermo Fisher Scientific or Jackson ImmunoResearch).

The fluorescent in situ hybridization (FISH) procedure for Arc and GFP was based on a previously published protocol (Daberkow et al., 2007). We used a full-length rat Arc ribonucleotide probe (rat and mouse Arc are 99% identical at the aa level) or EGFP (see cloning strategy above in “Plasmids”) as in the published protocol, but modified the protocol for use in cultured neurons and HEK cells instead of brain sections. Arc and GFP plasmids were linearized with Not1 and purified via standard phenol/chloroform extraction. The linearized antisense Arc or GFP were used to make a ribonucleotide probe that had DIG-UTP incorporated using a T7 DIG RNA labeling kit (Sigma-Aldrich), then purified with a G-50 spin column (GE Healthcare). Cells were washed once with 37° C. 4% sucrose/1×PBS, then fixed for 15 min with 4% sucrose/4% formaldehyde in 1×PBS. Cells were washed 335 min with 1×PBS, permeabilized in 0.2% Triton X-100 for 10 min, washed 235 min in 1×PBS, then 5 min with 2× saline-sodium citrate (SSC; 20×: 3 M NaCl, 300 mM citric acid trisodium salt dihydrate, pH 7). Cells were prehybridized in 1× prehybridization solution (Sigma-Aldrich) for 30 min. The DIG-labeled Arc or GFP ribonucleotide probe was diluted 1:3 with ddH2O, denatured at 90° C. for 5 min, put on wet ice for 2 min, then mixed with RNA hybridization buffer (23.75 mM Tris-HCl, 1.19 mM EDTA, 357 mM NaCl, 11.9% dextran sulfate, 1.19×Denhardt's solution (Thermo Fisher Scientific), 2.5% nuclease-free water, 60% formamide (Fisher Scientific, Hampton, N.H.)). The Arc probe (1:500) or GFP probe (1:750) was hybridized to the cultured cells at 56° C. for 16 h. The following day, cells underwent a series of washes to decrease background signal: 335 min 2×SSC, 15 min in RNase A (1:1000; Omega Bio-tek) at 37° C., 10 min 2×SSC at RT, 10 min 0.2×SSC at RT, 15 min 0.2×SSC at 56° C., 10 min 0.2×SSC at RT, 5 min TNT (0.1 M Tris-HCl, 0.15 M NaCl, 0.05% Tween-20, pH 7.5). Cells were then blocked in TNB (0.1 M Tris-HCl, 0.15 M NaCl, 0.5% w/v blocking reagent (Sigma-Aldrich), pH 7.5) with 2.5% sheep serum (Jackson ImmunoResearch) and 2.5% donkey serum for 30 min. In the primary antibody step, a DIG-HRP (1:1000; Sigma-Aldrich) and either MAP2 (1:2500; Abcam), Arc (1:500; custom-made), or Rab5 (1:500; BD Biosciences) antibody were diluted together in TNB with 2.5% sheep serum and 2.5% donkey serum and incubated on the cells for 1 h. After 335 min washes in TNT, the DIG-HRP signal was developed using a TSA Plus Cyanine 3 kit (1:50; PerkinElmer, Waltham, Mass.) for 30 min. Cells were washed for 5 min in TNT and 5 min in 1×PBS, then secondary antibody was diluted 1:750 in 5% donkey serum and 1×PBS and incubated on the cells for 1 h to detect MAP2, Arc, or Rab5. Nuclei were stained with DAPI (Thermo Fisher Scientific), then coverslips were mounted on glass slides with Fluoromount and dried overnight at RT

b. Imaging

Coverslips were imaged using a 60× oil objective on an Olympus FV1000 confocal microscope (Tokyo, Japan) and images were analyzed using ImageJ software (National Institutes of Health, Bethesda, Md.). Neurons included for analysis were selected in an unbiased manner by looking at MAP2 dendritic morphology for cell health. Coverslips were viewed blind to find the brightest immunofluorescence in each independent experiment, and this value was then used to set the image acquisition settings for that experiment. Images from all coverslips in that experiment were then acquired using the exact same settings.

c. Analysis of Dendritic Arc Protein and mRNA Expression

During analysis, images were blindly thresholded (to remove background fluorescence and to ensure images were analyzed in the linear range) to the brightest immunofluorescence in an individual experiment, and the same threshold was applied to all other images in that experiment. Integrated density (average pixel intensity x area) of two 30-mm dendritic segments/neuron was measured from each coverslip. In general, thick proximal dendritic branches were avoided in our analysis to control for potential differences in dendritic volume. The KO control group in each experiment, whether ICC or FISH, was set as “1,” and the integrated density values in the other groups were normalized to this and are displayed in the graphs as fold-change ±SEM. For representative images in the figures, the Smart look-up table (LUT) in ImageJ was applied to highlight differences in Arc expression between groups. Analysis of Arc/Rab5 colocalization Two 30-mm dendritic segments/neuron were selected for analysis of Arc protein or mRNA colocalization with Rab5 protein. The Arc channel and Rab5 channel were thresholded to the same value across all images. Using ImageJ, a mask was made of the thresholded section of dendrite for both Rab5 and Arc. The Arc mask was applied to the Rab5 mask and the number of overlapping puncta was quantified. The number of Arc particles overlapping Rab5 was divided by the total number of Arc particles in the stretch of dendrite to determine the Arc/Rab5 colocalization

d. Western Blots Immunoblotting and Analysis

Western blot samples were mixed with 4× Laemlli buffer (40% glycerol, 250 mM Tris, 4% SDS, 50 mM DTT, pH 6.8) and heated at 70° C. for 5 min. SDS-PAGE gel electrophoresis was used to separate protein samples. Separated samples were transferred to a nitrocellulose membrane (GE Healthcare). Following transfer, membranes were briefly stained with 0.1% Ponceau stain, then destained with 1% acetic acid to remove background, for imaging of total protein. Membranes were blocked in 5% milk+1× tris-buffered saline (TBS; 10×: 152.3 mM Tris-HCl, 46.2 mM Tris base, 1.5 M NaCl, pH 7.6) for 30 min at RT, then incubated in primary antibody in 1×TBS for either 1 h at RT or overnight at 4° C. Membranes were washed 3×10 min in 1×TBS, then incubated in an HRP-conjugated secondary antibody (Jackson ImmunoResearch) in block for 1 h at RT. After 3×10 min in 1×TBS, a chemiluminescent kit (Bio-Rad, Hercules, Calif.) was used to detect the protein bands, and the membranes were imaged on an Azure c300 gel dock (Azure Biosystems, Dublin, Calif.). Blots were analyzed and quantified using the Gel Analysis plugin in ImageJ.

e. Antibodies

Antibodies were used at the following concentrations: Arc (1:000; mouse monoclonal, Santa Cruz), Arc (1:000; rabbit polyclonal, custom, Protein Tech), ALIX (1:500; rabbit polyclonal, custom, provided by Dr. Wesley Sundquist), actin (1:1000; HRP-conjugated, Abcam), GFP (1:1000; chicken polyclonal, Ayes). All secondary antibodies were used at a dilution of 1:10,000 (HRP-conjugated goat anti-rabbit, goat anti-mouse, goat anti-chicken, Jackson ImmunoResearch). Coomassie Gels

Samples for analysis via SDS-PAGE were mixed with 4× Laemlli buffer and heated at 70° C. for 5 min. Protein samples were separated on 10% SDS gels. Gels were then stained with 0.1% Coomassie blue stain (0.1% w/v Coomassie blue, 50% methanol, 10% acetic acid, 40% water) for 30 min and destained overnight in destain solution (50% methanol, 10% acetic acid, 40% water). Gels were visualized using an Azure c300 gel dock under the auto-exposure setting on the visible channel. Gel exposures were analyzed and quantified using the Gel Analysis plugin in ImageJ.

viii. Immunoprecipitation

WT and Arc KO cortices were dissected out and homogenized in 150 mM NaCl, 50 mM Tris, 1% Triton X-100, 0.5% sodium deoxycholate, 0.05% SDS, pH 7.4 (IP lysis buffer), with protease inhibitor added fresh (Roche). Homogenates were pelleted at 200×g for 5 min at 4° C. to remove tissue debris. Supernatants were removed, diluted from 2 mL to 4 mL, and rocked at 4° C. for 10 min before being pelleted at 17,000×g for 10 min at 4° C. to remove insoluble material. Cleared supernatants were removed, a small aliquot was taken as the input, and the remainder used for immunoprecipitation. Supernatants were immunoprecipitated with either Arc antibody (rabbit polyclonal, custom-made; Protein Tech) or normal rabbit IgG (Santa Cruz Biotechnology, Santa Cruz, Calif.) at 1 mg/500 mL lysate for 2 h at 4° C. with gentle rocking. Following antibody incubation, a 10% volume of washed 50/50 Protein A bead slurry (Thermo Fisher Scientific) was added to the antibody/lysate mixture and incubated for an additional hour at 4° C. with rocking. Bead-antibody complexes were then pelleted briefly at low speed, supernatants were removed, and beads were washed three times with IP buffer. Washed beads were then resuspended in 200 mL IP buffer. With half of the bead slurry, protein was eluted from the beads with 17 mL 4× Laemlli buffer for 5 min at RT, then 50 mL IP buffer was added and the solution was removed from the beads into a new tube and heated at 70° C. for 5 min. The input (10% lysate volume) and 30 mL each of the IgG and antibody elutions were separated by SDS-PAGE on a 10% acrylamide gel and immunoblotted as described above. The bands for the input and IgG and Arc elutions were analyzed using the Gel Analysis plugin in ImageJ, and the data were represented graphically as a ratio of the signal from each elution over the input signal from each individual mouse. With the other half of the bead slurry, the IP buffer was adjusted to 1% SDS and 0.8 mg Proteinase K (New England Biolabs, Ipswich, Mass.) was added. Samples were then incubated at RT for 30 min with rocking and total RNA was extracted as described below.

ix. Chemical Crosslinking of Arc Proteins In Situ

Transfected HEK cells expressing myc-Arc-WT or a GFP control were briefly trypsinized, quenched with DMEM (Thermo Fisher Scientific), and pelleted. Media was removed and pelleted cells were then crosslinked with 0.4% formaldehyde in PBS for 10 min with rocking at RT. Cell suspensions were immediately quenched with Tris to a final concentration of 50 mM and repelleted. Supernatants were removed and cell pellets were then lysed with 150 mM NaCl, 50 mM Tris, 1% Triton X-100, pH 7.4 (lysis buffer) for 20 min at 4° C. with rocking. Lysates were cleared by centrifugation at 21,000×g for 10 min at 4° C. and cleared supernatants were then run on a 4%-8% gradient gel and analyzed via western blot with antibodies for Arc (mouse monoclonal, Santa Cruz) and GFP (chicken polyclonal, Ayes).

x. RNA extraction

For all samples, total RNA was extracted using TRIzol (Thermo Fisher Scientific). TRIzol-extracted samples were mixed 5:1 with chloroform, incubated at RT for 3 min, and pelleted at 12,000×g at 4° C. for 10 min. The resulting aqueous phase was taken and mixed 1:1 with isopropanol, incubated at RT, and pelleted at 12,000×g at 4° C. for 10 min. The resulting supernatant was removed and pellet washed with cold 75% ethanol. Washed pellets were then repelleted at 7500×g for 5 min at 4° C. The supernatant was removed and dried pellets were resuspended in ddH2O.

xi. RT-PCR

Total RNA concentrations were measured by A260/280 on a Nanodrop (Thermo Scientific). Reverse transcription reactions were carried out using a High Capacity cDNA Reverse Transcription Kit (Applied Biosystems, Foster City, Calif.) with 100-200 ng of RNA as template. Resulting cDNAs were amplified using rat Arc, GAPDH primer sets for 35 cycles with a 60° C. annealing temperature. Resulting PCR products were analyzed on 1.5% agarose gels stained with SYBR Safe (Thermo Fisher Scientific). Rat Arc primers: Fwd, ACCATATGACCACCGGCGGC (SEQ ID NO:5); Rev, TCCAGCATCTCAGCTCGGCAC (SEQ ID NO:6). GAPDH primers: Fwd, CATGGCCTTCCGTGTTCCTA (SEQ ID NO:7); Rev, GCCTGCTTCACCACCTTCTT (SEQ ID NO:8). RT-PCR gels were quantified using the ImageJ gel analyzer tool.

xii. qRT-PCR

To determine the amount of RNA associated with Arc protein, quantitative RT-PCR was performed on mRNA prepared from 1: whole mouse cortices immunoprecipitated with Arc and IgG protein, 2: EV fractions prepared from HEK cells (see below, “Extracellular vesicle purification”), and 3: lysate and purified protein from bacteria (BL21, Thermo Fisher Scientific) transfected with rat Arc plasmid (pGEX-GST-ArcFL). Some samples were treated with RNase (25 mg, RNase A, Thermo Fisher Scientific) to determine if the mRNA associated with Arc protein was protected from degradation relative to exogenously added GFP antisense RNA (generating using T7 RNA polymerase from linearized pBluescript-SKII-GFP). Preparation 1: Mice were sacrificed after 24 h of dark-housing and 2 h of enriched environment. Whole cortices were dissected and homogenized in IP lysis buffer as described above. After immunoprecipitation, bead slurry was incubated in guanidine thiocyanate containing RLT lysis buffer and column purification of RNA was performed using QIAGEN RNeasy Micro Kit (QIAGEN, Hilden, Germany). Total eluate was used for reverse transcription using High Capacity cDNA Reverse Transcription Kit with 50 U of Multiscribe Reverse Transcriptase and random oligo primers (Thermo Fisher Scientific). Preparations 2 and 3: total RNA was extracted using TRIzol (Thermo Fisher Scientific) as described above (“RNA extraction”). Reverse transcription reactions (25° C. for 10 min, 37° C. for 2 h, 85° C. for 5 min) were carried out using a High Capacity cDNA Reverse Transcription Kit. Resulting cDNA was prepared for qPCR using PowerUp SYBRgreen Master Mix (Thermo Fisher Scientific) in a 96-well plate with primers against rat Arc, GAPDH and asnA (see above, “RT-PCR”; asnA primers: Fwd, GCGTGGATGCCGACACGTTG (SEQ ID NO:10); Rev, ATACCGCCGCCGATGGTCTG (SEQ ID NO:11)). qPCR was performed on a QuantStudio 3 Real Time PCR System (Thermo Fisher Scientific) using the following protocol: Pre-incubation: 50° C. for 2 min, 95° C. for 2 min. Amplification: 40 cycles of 95° C. for 15 s, 60° C. for 15 s, and 72° C. for 1 min. Melt curve: 95° C. for 1 s, 60° C. for 20 s, continuous ramp at 0.15° C./s up to 95° C. Ct values of greater than 30 were considered undetectable. Differences in expression were determined using the standard curve method, where a standard DNA sample was serially diluted (10-fold), analyzed for the gene of interest, and the linear equation calculated. The resulting linear equation was used to determine where the Ct values of test samples fell within the standard curve and the result was transformed (log 10) to reflect the dilution of the standard sample. Differences were calculated measuring the fold-change from the average of the control values for any given group (test/average control).

xiii. Extracellular Vesicle Purification

Extracellular vesicles (EVs) were purified from HEK cell and primary neuronal cultures as previously described (Lachenal et al., 2011). Media was spun successively at 2,000 and 20,000×g to remove dead cells and debris, and then at 100,000×g to pellet EVs. The crude EV pellet following the initial high-speed spin was resuspended in cold PBS and repelleted at 100,000×g for 1 h at 4° C. in an SW41 rotor. The washed EV pellet was further purified by centrifugation over a 10%-20% sucrose-PBS gradient at 100,000×g overnight at 4° C. The resulting pellet was washed in cold PBS to remove excess sucrose and then repelleted at 100,000×g for 1 h at 4° C. The final, washed pellet was resuspended in PBS and used for downstream analysis with EM, western blotting, and neuron treatments. Trypsin digestion and RNase assays Trypsin was added to prArc and EVs at 0.05 mg/mL for 30 min at RT followed by addition of 1 mM PMSF for 10 min to inactivate trypsin. Untreated and trypsin-treated samples were then analyzed by western blot. RNase A was added to WT neuron lysates and EVs at 50 mg/mL for 15 min at 37° C. Untreated and RNase-treated samples for RT-PCR were then directly extracted with TRIzol. Trypsin digestion and RNase assays

Trypsin was added to prArc and EVs at 0.05 mg/ml for 30 min at RT followed by addition of 1 mM PMSF for 10 min to inactivate trypsin. Untreated and trypsin-treated samples were then analyzed by western blot. RNase A was added to WT neuron lysates and EVs at 50 ug/ml for 15 min at 37° C. followed. Untreated and RNase-treated samples for RT-PCR were then directly extracted with Trizol.

xiv. Immunogold Labeling

Immunogold labeling was performed with modifications as previously described (Korkut et al., 2013). Samples were fixed overnight in 2% formaldehyde at 4° C. with gentle rocking. Samples were then applied to glow discharged Formvar copper mesh grids (Ted Pella) and allowed to adhere at room temperature for 10 min. Samples were then quenched by 3 washes of 0.1 M Tris, pH 7.4. Samples were then permeabilized for 10 min at RT, blocked, and stained for Arc (1:500; custom-made). 5 nm gold-conjugated secondary antibodies were used for staining without silver enhancement. Following antibody labeling, grids were negative stained as described above

xv. Statistics

Two-way ANOVA with or without repeated-measures (with post hoc Sidak's tests) or two-tailed unpaired t-tests were performed using GraphPad Prism (GraphPad Software, San Diego, Calif.) or JMP Pro statistical software (SAS; Cary, N.C.). Significance was set at p<0.05. All data shown are representative of at least two experimental replicates. Details of the statistics (N, number of experimental replicates, description of how the data are displayed) can be found in figure legends and/or the Results section. Those skilled in the art will recognize, or be able to ascertain using no more than routine experimentation, many equivalents to the specific embodiments of the method and compositions described herein. Such equivalents are intended to be encompassed by the following claims.

1-43. (canceled)

44. An Arc capsid comprising a heterologous nucleic acid.

45. The Arc capsid of claim 44, wherein the heterologous nucleic acid is a therapeutic nucleic acid.

46. The Arc capsid of claim 44, wherein the heterologous nucleic acid is an mRNA, siRNA or shRNA.

47. An extracellular vesicle comprising the Arc capsid of claim 44.

48. A method of making the Arc capsid of claim 1 comprising preparing a solution comprising Arc protein and heterologous nucleic acid and increasing the salt concentration of the solution to a range of 100 mM to 500 mM

49. The method of claim 48, wherein the salt is NaCl or NaPO₄.

50. A method of making the Arc capsid of claim 44 comprising administering a nucleic acid encoding a heterologous RNA to a host cell.

51. The method of claim 50, further comprising administering a nucleic acid encoding an Arc protein to the host cell.

52. A method of making the extracellular vesicle of claim 47 comprising administering a nucleic acid encoding a heterologous RNA to a host cell and isolating the extracellular vesicle from the host cell.

53. The method of claim 52, further comprising administering a nucleic acid encoding an Arc protein to the host cell.

54. A host cell comprising the Arc capsid of claim 44.

55. The host cell of claim 54, wherein the host cell is a neuron.

56. The host cell of claim 54, wherein the host cell comprises a recombinant nucleic acid encoding an Arc protein.

57. A method of delivering a nucleic acid to a target cell comprising administering the Arc capsid of claim 44 to the target cell.

58. The method of claim 57, wherein the nucleic acid is an mRNA encoding a therapeutic protein.

59. The method of claim 57, wherein the nucleic acid is an siRNA or shRNA.

60. The method of claim 57, wherein the target cell is a neuron.

61. The method of claim 57, wherein the target cell is a human cell.

62. A method of delivering an RNA to a target cell comprising administering the extracellular vesicle of claim 47 to the target cell.

63. The method of claim 62, wherein the nucleic acid is an mRNA encoding a therapeutic protein.

64. The method of claim 63, wherein the therapeutic protein is expressed.

65. The method of claim 62, wherein the nucleic acid is an siRNA or shRNA.

66. The method of claim 62, wherein the target cell internalizes the extracellular vesicle by endocytosis.

67. The method of claim 62, wherein the nucleic acid is released into the target cell.

68. The method of claim 62, wherein the target cell is a neuron.

69. The method of claim 62, wherein the target cell is a human cell.

70. A method of delivering an RNA to a target cell comprising administering a nucleic acid encoding an Arc protein to a host cell, and administering a nucleic acid encoding a heterologous RNA to the host cell, wherein the Arc capsid comprising the heterologous RNA forms within the host cell.

71. The method of claim 70, wherein the nucleic acid is an mRNA encoding a therapeutic protein.

72. The method of claim 70, wherein the nucleic acid is an siRNA or shRNA.

73. The method of claim 70, further comprising contacting the host cell to the target cell.

74. The method of claim 70, wherein the target cell is a neuron.

75. The method of claim 70, wherein the target cell is a human cell.
